SEQUENCE LISTING (63 sequences)

SEQ ID NO: 1 - DNA, 1365 bp, synthetic construct
  misc_feature 1..1365: note = Synthetic
  source 1..1365: mol_type = other DNA
gcgaagctttgagccaccatggaaaccccagcgcagcttctcttcctcctgctactctggctcccagataccaccggtgacatccagatgactcagtctccagcctccctatctgcatctgtgggagaaactgtcaccatcacatgtcgagcaagtgagaagatttacagttatttagcatggtatcagcagaaacagggaaaatctcctcagctcctggtctataacgcaaaaaccttagcagaaggtgtgccatcaaggttcagtgtcagtggatcaggcacacagttttctctgaggatcaacagcctgcagcctgaagattttgggacttattactgtcaacatcattttggttctcctcggacgttcggtggaggcaccaaactggaaatcaaagatctcggaggaggtggctcaggtggtggaggatctggaggaggtgggagtggtggaggtggttctaccggtgaggtccagctgcaacagtctggacctgaactggtgaagcctggagcttcaatgaagatatcctgcaaggcttctggttactcattcactggctacaccatgaactgggtgaagcagagccatggaaagacccttgaatggattggacttattaatccttacaatggtgttactagctacaaccagaagttcaaggacaaggccacattaactgtagacaagtcatccagcacagcctacatggaactcctcagtctgacatctgaggactctgcaatctattactgtgcaagagactataattacgactactttgactactggggccaaggcaccactctcacagtctcctcaggaggaggtggctcaggtggtggaggatctggaggaggtgggagtggtggaggtggttctatggacattgacccgtataaagaatttggagcttctgtggagttactctcttttttgccttctgacttctttccttctattcgagatctcctcgacaccgcctctgctctgtatcgggaggccttagagtctccggaacattgttcacctcaccatacagcactcaggcaagctattctgtgttggggtgagttgatgaatctggccacctgggtgggaagtaatttggaagacccagcatccagggaattagtagtcagctatgtcaatgttaatatgggcctaaaaattagacaactattgtggtttcacatttcctgccttacttttggaagagaaactgtccttgagtatttggtgtcttttggagtgtggattcgcactcctcccgctgccagaccaccaaatgcccctatcttatcaacacttccggaaactactgttgttcaccaccatcatcatcattgataaggatccgcg

SEQ ID NO: 2 - protein, 444 aa, synthetic construct
  REGION 1..444: note = Synthetic
  VARIANT 439..444: note = optionally absent
METPAQLLFLLLLWLPDTTGDIQMTQSPASLSASVGETVTITCRASEKIYSYLAWYQQKQGKSPQLLVYNAKTLAEGVPSRFSVSGSGTQFSLRINSLQPEDFGTYYCQHHFGSPRTFGGGTKLEIKDLGGGGSGGGGSGGGGSGGGGSTGEVQLQQSGPELVKPGASMKISCKASGYSFTGYTMNWVKQSHGKTLEWIGLINPYNGVTSYNQKFKDKATLTVDKSSSTAYMELLSLTSEDSAIYYCARDYNYDYFDYWGQGTTLTVSSGGGGSGGGGSGGGGSGGGGSMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAARPPNAPILSTLPETTVVHHHHHH

SEQ ID NO: 3 - protein, 424 aa, synthetic construct
  REGION 1..424: note = Synthetic
  VARIANT 419..424: note = optionally absent
DIQMTQSPASLSASVGETVTITCRASEKIYSYLAWYQQKQGKSPQLLVYNAKTLAEGVPSRFSVSGSGTQFSLRINSLQPEDFGTYYCQHHFGSPRTFGGGTKLEIKDLGGGGSGGGGSGGGGSGGGGSTGEVQLQQSGPELVKPGASMKISCKASGYSFTGYTMNWVKQSHGKTLEWIGLINPYNGVTSYNQKFKDKATLTVDKSSSTAYMELLSLTSEDSAIYYCARDYNYDYFDYWGQGTTLTVSSGGGGSGGGGSGGGGSGGGGSMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAARPPNAPILSTLPETTVVHHHHHH

SEQ ID NO: 4 - protein, 20 aa, synthetic construct
  REGION 1..20: note = Synthetic
GGGGSGGGGSGGGGSGGGGS

SEQ ID NO: 5 - protein, 444 aa, synthetic construct
  REGION 1..444: note = Synthetic
  VARIANT 439..444: note = optionally absent
METPAQLLFLLLLWLPDTTGDIQMTQSPASLSASVGETVTITCRASEKIYSYLAWYQQKQGKSPQLLVYNAKTLAEGVPSRFSVSGSGTQFSLRINSLQPEDFGTYYCQHHFGSPRTFGGGTKLEIKDLGGGGSGGGGSGGGGSGGGGSTGEVQLQQSGPELVKPGASMKISCKASGYSFTGYTMNWVKQSHGKTLEWIGLINPYNGVTSYNQKFKDKATLTVDKSSSTAYMELLSLTSEDSAIYYCARDYNYDYFDYWGQGTTLTVSSGGGGSGGGGSGGGGSGGGGSMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVHHHHHH

SEQ ID NO: 6 - protein, 424 aa, synthetic construct
  REGION 1..424: note = Synthetic
  VARIANT 419..424: note = optionally absent
DIQMTQSPASLSASVGETVTITCRASEKIYSYLAWYQQKQGKSPQLLVYNAKTLAEGVPSRFSVSGSGTQFSLRINSLQPEDFGTYYCQHHFGSPRTFGGGTKLEIKDLGGGGSGGGGSGGGGSGGGGSTGEVQLQQSGPELVKPGASMKISCKASGYSFTGYTMNWVKQSHGKTLEWIGLINPYNGVTSYNQKFKDKATLTVDKSSSTAYMELLSLTSEDSAIYYCARDYNYDYFDYWGQGTTLTVSSGGGGSGGGGSGGGGSGGGGSMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVHHHHHH

SEQ ID NO: 7 - protein, 16 aa, synthetic construct
  REGION 1..16: note = Synthetic
DFLVIYIEEAHASDGW

SEQ ID NO: 8 - protein, 474 aa, synthetic construct
  REGION 1..474: note = Synthetic
  VARIANT 469..474: note = optionally absent
METPAQLLFLLLLWLPDTTGDIQMTQSPASLSASVGETVTITCRASEKIYSYLAWYQQKQGKSPQLLVYNAKTLAEGVPSRFSVSGSGTQFSLRINSLQPEDFGTYYCQHHFGSPRTFGGGTKLEIKDLGGGGSGGGGSGGGGSGGGGSTGEVQLQQSGPELVKPGASMKISCKASGYSFTGYTMNWVKQSHGKTLEWIGLINPYNGVTSYNQKFKDKATLTVDKSSSTAYMELLSLTSEDSAIYYCARDYNYDYFDYWGQGTTLTVSSGGGGSGGGGSGGGGSGGGGSMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAARPPNAPILSTLPETTVVRRRGRSPRRRTPSPRRRRSQSPRRRRSQSRHHHHHH

SEQ ID NO: 9 - protein, 454 aa, synthetic construct
  REGION 1..454: note = Synthetic
  VARIANT 449..454: note = optionally absent
DIQMTQSPASLSASVGETVTITCRASEKIYSYLAWYQQKQGKSPQLLVYNAKTLAEGVPSRFSVSGSGTQFSLRINSLQPEDFGTYYCQHHFGSPRTFGGGTKLEIKDLGGGGSGGGGSGGGGSGGGGSTGEVQLQQSGPELVKPGASMKISCKASGYSFTGYTMNWVKQSHGKTLEWIGLINPYNGVTSYNQKFKDKATLTVDKSSSTAYMELLSLTSEDSAIYYCARDYNYDYFDYWGQGTTLTVSSGGGGSGGGGSGGGGSGGGGSMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAARPPNAPILSTLPETTVVRRRGRSPRRRTPSPRRRRSQSPRRRRSQSRHHHHHH

SEQ ID NO: 10 - protein, 20 aa, synthetic construct
  REGION 1..20: note = Synthetic
QGPKEPFRDYVDRFYKTLRA

SEQ ID NO: 11 - protein, 474 aa, synthetic construct
  REGION 1..474: note = Synthetic
  VARIANT 469..474: note = optionally absent
METPAQLLFLLLLWLPDTTGDIQMTQSPASLSASVGETVTITCRASEKIYSYLAWYQQKQGKSPQLLVYNAKTLAEGVPSRFSVSGSGTQFSLRINSLQPEDFGTYYCQHHFGSPRTFGGGTKLEIKDLGGGGSGGGGSGGGGSGGGGSTGEVQLQQSGPELVKPGASMKISCKASGYSFTGYTMNWVKQSHGKTLEWIGLINPYNGVTSYNQKFKDKATLTVDKSSSTAYMELLSLTSEDSAIYYCARDYNYDYFDYWGQGTTLTVSSGGGGSGGGGSGGGGSGGGGSMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRHHHHHH

SEQ ID NO: 12 - protein, 454 aa, synthetic construct
  REGION 1..454: note = Synthetic
  VARIANT 449..454: note = optionally absent
DIQMTQSPASLSASVGETVTITCRASEKIYSYLAWYQQKQGKSPQLLVYNAKTLAEGVPSRFSVSGSGTQFSLRINSLQPEDFGTYYCQHHFGSPRTFGGGTKLEIKDLGGGGSGGGGSGGGGSGGGGSTGEVQLQQSGPELVKPGASMKISCKASGYSFTGYTMNWVKQSHGKTLEWIGLINPYNGVTSYNQKFKDKATLTVDKSSSTAYMELLSLTSEDSAIYYCARDYNYDYFDYWGQGTTLTVSSGGGGSGGGGSGGGGSGGGGSMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRHHHHHH

SEQ ID NO: 13 - protein, 179 aa, synthetic construct
  REGION 1..179: note = Synthetic
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSR

SEQ ID NO: 14 - protein, 179 aa, synthetic construct
  REGION 1..179: note = Synthetic
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAARPPNAPILSTLPETTVVRRRGRSPRRRTPSPRRRRSQSPRRRRSQSR

SEQ ID NO: 15 - protein, 149 aa, synthetic construct
  REGION 1..149: note = Synthetic
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVV

SEQ ID NO: 16 - protein, 149 aa, synthetic construct
  REGION 1..149: note = Synthetic
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAARPPNAPILSTLPETTVV

SEQ ID NO: 17 - protein, 159 aa, synthetic construct
  REGION 1..159: note = Synthetic
SKLCLGWLWGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVV

SEQ ID NO: 18 - protein, 454 aa, synthetic construct
  REGION 1..454: note = Synthetic
  VARIANT 449..454: note = optionally absent
METPAQLLFLLLLWLPDTTGDIQMTQSPASLSASVGETVTITCRASEKIYSYLAWYQQKQGKSPQLLVYNAKTLAEGVPSRFSVSGSGTQFSLRINSLQPEDFGTYYCQHHFGSPRTFGGGTKLEIKDLGGGGSGGGGSGGGGSGGGGSTGEVQLQQSGPELVKPGASMKISCKASGYSFTGYTMNWVKQSHGKTLEWIGLINPYNGVTSYNQKFKDKATLTVDKSSSTAYMELLSLTSEDSAIYYCARDYNYDYFDYWGQGTTLTVSSGGGGSGGGGSGGGGSGGGGSSKLCLGWLWGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVHHHHHH

SEQ ID NO: 19 - protein, 434 aa, synthetic construct
  REGION 1..434: note = Synthetic
  VARIANT 429..434: note = optionally absent
DIQMTQSPASLSASVGETVTITCRASEKIYSYLAWYQQKQGKSPQLLVYNAKTLAEGVPSRFSVSGSGTQFSLRINSLQPEDFGTYYCQHHFGSPRTFGGGTKLEIKDLGGGGSGGGGSGGGGSGGGGSTGEVQLQQSGPELVKPGASMKISCKASGYSFTGYTMNWVKQSHGKTLEWIGLINPYNGVTSYNQKFKDKATLTVDKSSSTAYMELLSLTSEDSAIYYCARDYNYDYFDYWGQGTTLTVSSGGGGSGGGGSGGGGSGGGGSSKLCLGWLWGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVHHHHHH

SEQ ID NO: 20 - protein, 453 aa, synthetic construct
  REGION 1..453: note = Synthetic
  VARIANT 448..453: note = optionally absent
MQLFPLCLIISCSCPTVQASKLCLGWLWGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVGGGGSGGGGSGGGGSGGGGSDIQMTQSPASLSASVGETVTITCRASEKIYSYLAWYQQKQGKSPQLLVYNAKTLAEGVPSRFSVSGSGTQFSLRINSLQPEDFGTYYCQHHFGSPRTFGGGTKLEIKDLGGGGSGGGGSGGGGSGGGGSTGEVQLQQSGPELVKPGASMKISCKASGYSFTGYTMNWVKQSHGKTLEWIGLINPYNGVTSYNQKFKDKATLTVDKSSSTAYMELLSLTSEDSAIYYCARDYNYDYFDYWGQGTTLTVSSHHHHHH

SEQ ID NO: 21 - protein, 434 aa, synthetic construct
  REGION 1..434: note = Synthetic
  VARIANT 429..434: note = optionally absent
SKLCLGWLWGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVGGGGSGGGGSGGGGSGGGGSDIQMTQSPASLSASVGETVTITCRASEKIYSYLAWYQQKQGKSPQLLVYNAKTLAEGVPSRFSVSGSGTQFSLRINSLQPEDFGTYYCQHHFGSPRTFGGGTKLEIKDLGGGGSGGGGSGGGGSGGGGSTGEVQLQQSGPELVKPGASMKISCKASGYSFTGYTMNWVKQSHGKTLEWIGLINPYNGVTSYNQKFKDKATLTVDKSSSTAYMELLSLTSEDSAIYYCARDYNYDYFDYWGQGTTLTVSSHHHHHH

SEQ ID NO: 22 - protein, 25 aa, synthetic construct
  REGION 1..25: note = Synthetic
HMKQLDVEELSNYHLNVARLKVGER

SEQ ID NO: 23 - protein, 33 aa, synthetic construct
  REGION 1..33: note = Synthetic
GVTQLMREMLQLIKFQFSLNYQEESLSYQRLVT

SEQ ID NO: 24 - protein, 7 aa, synthetic construct
  REGION 1..7: note = Synthetic
EVSALEK

SEQ ID NO: 25 - protein, 7 aa, synthetic construct
  REGION 1..7: note = Synthetic
KVSALKE

SEQ ID NO: 26 - protein, 15 aa, synthetic construct
  REGION 1..15: note = Synthetic
SKLCLGWLWGMDIDP

SEQ ID NO: 27 - protein, 15 aa, synthetic construct
  REGION 1..15: note = Synthetic
LGWLWGMDIDPYKEF

SEQ ID NO: 28 - protein, 15 aa, synthetic construct
  REGION 1..15: note = Synthetic
WGMDIDPYKEFGASV

SEQ ID NO: 29 - protein, 15 aa, synthetic construct
  REGION 1..15: note = Synthetic
IDPYKEFGASVELLS

SEQ ID NO: 30 - protein, 15 aa, synthetic construct
  REGION 1..15: note = Synthetic
KEFGASVELLSFLPS

SEQ ID NO: 31 - protein, 15 aa, synthetic construct
  REGION 1..15: note = Synthetic
ASVELLSFLPSDFFP

SEQ ID NO: 32 - protein, 15 aa, synthetic construct
  REGION 1..15: note = Synthetic
LLSFLPSDFFPSIRD

SEQ ID NO: 33 - protein, 15 aa, synthetic construct
  REGION 1..15: note = Synthetic
LPSDFFPSIRDLLDT

SEQ ID NO: 34 - protein, 15 aa, synthetic construct
  REGION 1..15: note = Synthetic
FFPSIRDLLDTASAL

SEQ ID NO: 35 - protein, 15 aa, synthetic construct
  REGION 1..15: note = Synthetic
IRDLLDTASALYREA

SEQ ID NO: 36 - protein, 15 aa, synthetic construct
  REGION 1..15: note = Synthetic
LDTASALYREALESP

SEQ ID NO: 37 - protein, 15 aa, synthetic construct
  REGION 1..15: note = Synthetic
SALYREALESPEHCS

SEQ ID NO: 38 - protein, 15 aa, synthetic construct
  REGION 1..15: note = Synthetic
REALESPEHCSPHHT

SEQ ID NO: 39 - protein, 15 aa, synthetic construct
  REGION 1..15: note = Synthetic
ESPEHCSPHHTALRQ

SEQ ID NO: 40 - protein, 15 aa, synthetic construct
  REGION 1..15: note = Synthetic
HCSPHHTALRQAILC

SEQ ID NO: 41 - protein, 15 aa, synthetic construct
  REGION 1..15: note = Synthetic
HHTALRQAILCWGEL

SEQ ID NO: 42 - protein, 15 aa, synthetic construct
  REGION 1..15: note = Synthetic
LRQAILCWGELMNLA

SEQ ID NO: 43 - protein, 15 aa, synthetic construct
  REGION 1..15: note = Synthetic
ILCWGELMNLATWVG

SEQ ID NO: 44 - protein, 15 aa, synthetic construct
  REGION 1..15: note = Synthetic
GELMNLATWVGSNLE

SEQ ID NO: 45 - protein, 15 aa, synthetic construct
  REGION 1..15: note = Synthetic
NLATWVGSNLEDPAS

SEQ ID NO: 46 - protein, 15 aa, synthetic construct
  REGION 1..15: note = Synthetic
WVGSNLEDPASRELV

SEQ ID NO: 47 - protein, 15 aa, synthetic construct
  REGION 1..15: note = Synthetic
NLEDPASRELVVSYV

SEQ ID NO: 48 - protein, 15 aa, synthetic construct
  REGION 1..15: note = Synthetic
PASRELVVSYVNVNM

SEQ ID NO: 49 - protein, 15 aa, synthetic construct
  REGION 1..15: note = Synthetic
ELVVSYVNVNMGLKI

SEQ ID NO: 50 - protein, 15 aa, synthetic construct
  REGION 1..15: note = Synthetic
SYVNVNMGLKIRQLL

SEQ ID NO: 51 - protein, 15 aa, synthetic construct
  REGION 1..15: note = Synthetic
VNMGLKIRQLLWFHI

SEQ ID NO: 52 - protein, 15 aa, synthetic construct
  REGION 1..15: note = Synthetic
LKIRQLLWFHISCLT

SEQ ID NO: 53 - protein, 15 aa, synthetic construct
  REGION 1..15: note = Synthetic
QLLWFHISCLTFGRE

SEQ ID NO: 54 - protein, 15 aa, synthetic construct
  REGION 1..15: note = Synthetic
FHISCLTFGRETVLE

SEQ ID NO: 55 - protein, 15 aa, synthetic construct
  REGION 1..15: note = Synthetic
CLTFGRETVLEYLVS

SEQ ID NO: 56 - protein, 15 aa, synthetic construct
  REGION 1..15: note = Synthetic
GRETVLEYLVSFGVW

SEQ ID NO: 57 - protein, 15 aa, synthetic construct
  REGION 1..15: note = Synthetic
VLEYLVSFGVWIRTP

SEQ ID NO: 58 - protein, 15 aa, synthetic construct
  REGION 1..15: note = Synthetic
LVSFGVWIRTPPAYR

SEQ ID NO: 59 - protein, 15 aa, synthetic construct
  REGION 1..15: note = Synthetic
GVWIRTPPAYRPPNA

SEQ ID NO: 60 - protein, 15 aa, synthetic construct
  REGION 1..15: note = Synthetic
RTPPAYRPPNAPILS

SEQ ID NO: 61 - protein, 15 aa, synthetic construct
  REGION 1..15: note = Synthetic
AYRPPNAPILSTLPE

SEQ ID NO: 62 - protein, 15 aa, synthetic construct
  REGION 1..15: note = Synthetic
PNAPILSTLPETTVV

SEQ ID NO: 63 - protein, 12 aa, synthetic construct
  REGION 1..12: note = Synthetic
ADFLYIEAHDGW

(19) United States
(12) Patent Application Publication
(10) Pub. No.: US 2024/0123061 A1
(43) Pub. Date: Apr. 18, 2024

(54) Therapeutic Vaccine for Hepatitis B Virus (HBV) using the HBV Core Antigen

(71) Applicants: University of Washington, Seattle, WA (US); ABACUS BIOSCIENCE, INC., Sammamish, WA (US)

(72) Inventors: Edward A. CLARK, Seattle, WA (US); Deborah L. FULLER, Seattle, WA (US); Che-Leung LAW, Seattle, WA (US); Amanda MAK, Seattle, WA (US)

(21) Appl. No.: 18/533,676

(22) Filed: Dec. 8, 2023

Related U.S. Application Data
(62) Division of application No. 17/721,554, filed on Apr. 15, 2022, now Pat. No. 11,883,488.
Application No. 17/721,554 is a division of application No. 16/088,386, filed on Sep. 25, 2018, now Pat. No. 11,324,819.
(60) Provisional application No. 62/319,160, filed on Apr. 6, 2016.

(51) Int. Cl. A61K 39/29; A61K 39/00; A61K 39/39; C07K 16/28; C12N 7/00

(52) U.S. Cl. CPC A61K 39/292; A61K 39/001129; A61K 39/39; C07K 16/2896; C12N 7/00; C07K 2317/524; C07K 2317/526; C07K 2317/56; C07K 2317/622; C07K 2319/00; C12N 2730/10134

(57) ABSTRACT

Provided herein are compositions of CD1280 binding proteins and a Hepatitis B virus core antigen (HBcAg) and/or a Hepatitis B virus E antigen (HBeAg), or antigenic fragments or mutants thereof, attached to the CD180 binding protein, and methods for using the compositions to treat or limit the development of hepatitis-B virus (HBV)-related disorders.

### STATEMENT OF GOVERNMENT RIGHTS

This invention was made with government support under National Institute of Allergy & Infectious Diseases grant number AI044257. The government has certain rights in the invention.

### SEQUENCE LISTING

A computer readable form of the Sequence Listing is filed with this application by electronic submission and is incorporated into this application by reference in its entirety. The Sequence Listing is contained in the file created on Dec. 6, 2023 having the file name “16-1443-WO-US-DIV2.xml” and is 91,069 bytes in size.

### BACKGROUND OF THE INVENTION

In spite of the availability of prophylactic Hepatitis B virus (HBV) vaccines, HBV infection remains a very significant global health problem in both industrialized and developing nations; it is second only to tobacco as a cause of cancer. There is a clear unmet need for a therapeutic HBV vaccine for patients chronically infected with HBV (CHB). 10-30% of those vaccinated with marketed HBV vaccines do not respond either due to genetic factors, or non-compliance (failure to return for a series of 3 vaccinations). Only 37% of individuals vaccinated once with a licensed HBV vaccine are protected; even after three vaccinations, which are difficult to achieve, many people do not respond effectively. There is no effective vaccine for the 400 million people chronically infected with HBV, including asymptomatic HBV carriers. The drugs currently used to treat CHB patients are problematic. Sustained antiviral responses are rarely achieved and the currently available therapies can lead to viral resistance and produce side effects in many CHB patients.

### SUMMARY OF THE INVENTION

In one aspect, the invention provides compositions, comprising:
(a) a CD180 binding protein; and
(b) a Hepatitis B virus core antigen (HBcAg) and/or a Hepatitis B virus E antigen (HBeAg), or antigenic fragments or mutants thereof, attached to the CD180 binding protein.

In one embodiment, the CD180 binding protein is an anti-CD180 antibody or antibody fragment, such as an anti-CD180 monoclonal antibody or antibody fragment. In another embodiment, the CD180 binding protein comprises a single chain (sc) recombinant protein, wherein the sc recombinant protein comprises:
(i) a variable heavy (VH) chain region of an anti-CD180 antibody; and
(ii) a variable light (VL) chain region of an anti-CD180 antibody;

wherein the HBcAg and/or the HBeAg is located at the N-terminus or the C-terminus of the sc recombinant protein.

In one embodiment, the VH and VL chain regions are from an anti-human CD180 antibody, such as an anti-human CD180 monoclonal antibody. In another embodiment, the sc recombinant protein does not include any other immunoglobulin domains. In a futher embodiment, the sc recombinant protein further comprises:
(iii) CH2 and CH3 domains from an immunoglobulin (Ig), such as a human Ig, or functional mutants thereof, wherein the CH2 and CH3 domains are located C-terminal to the VH and VL domains.

For example, the sc recombinant protein may comprise CH2 and CH3 domains from IgG1, such as human IgG1, or functional mutants thereof. In one embodiment, the VL chain region is located N-terminal to the VH chain region. In another embodiment, the VH chain region is located N-terminal to the VL chain region. In a further embodiment, the CD180 binding protein competes for binding to CD180 with monoclonal antibody G28-8.

In another embodiment, the HBcAg and/or the HBeAg is/are capable of multimerizing, such as dimerizing. In one embodiment, the HBcAg and/or the HBeAg comprise a multimerization domain, such as a dimerization domain. In various further embodiments, the HBcAg and/or the HBeAg comprises a polypeptide at least 90% identical over its length to the amino acid sequence of SEQ ID NO:13, 14, 15, 16, or 17. In a further embodiment, the composition comprises a polypeptide at least 90% identical over its length to the amino acid sequence of SEQ ID NO:2, 3, 5, 6, 8, 9, 11, 12, 18, 19, 20, or 21.

In one embodiment, the composition further comprises an adjuvant, including but not limited to toll-like receptor 4 (TLR4) agonist, a toll-like receptor 7 (TLR7) agonist, a toll-like receptor 8 (TLR8) agonist, a toll-like receptor 9 (TLR9) agonist, alum-containing adjuvant, monophosphoryl lipid A, oil-in-water emulsion, and a-tocopherol, squalene and polysorbate 80 in an oil-in-water emulsion.

In further aspects, the invention provides isolated nucleic acids encoding a composition of any embodiment or combination of embodiments of the invention, expression vectors comprising the isolated nucleic acids of the invention operatively linked to a suitable control sequence, and recombinant host cell comprising the expression vectors of the invention.

In another aspect, the invention provides pharmaceutical compositions comprising the composition, isolated nucleic acid, or expression vector of any embodiment, in combination with a pharmaceutically acceptable carrier.

In a further aspect, the invention provides methods for treating or limiting the development of a hepatitis-B virus (HBV)-related disorder, comprising administering to an individual in need thereof an amount effective to treat or limit development of the HBV-related disorder of the composition, isolated nucleic acid, expression vector, or the pharmaceutical composition of any embodiment of the invention.

### BRIEF DESCRIPTION OF THE FIGURES

FIG. 1. Design of G28-8LH-HBcAg-Y132A-His protein.

FIG. 2. Characterization of recombinant G28-8LH-HBcAgY132A-His. G28-8LH-HBcAgY132A-His was transiently expressed in CHO cells. Culture supernatant was passed over a Ni2+ affinity chromatography column. Bound G28-8LH-HBcAgY132A-His was eluted with imidazole. Eluted protein (E) was characterized by reducing SDS-PAGE and western blotting using an anti-6×-His sera.

FIG. 3. Binding of recombinant G28-8LH-HBcAgY132A-His to human B cells detected by flow cytometry. A Direct binding to human CD20+ tonsillar B cells. B Competition binding assay between G28-8LH-HBcAgY13A-His and FITC-labeled parent G28-8 antibody.

FIG. 4. Recombinant G28-8LH-HBcAgY132A-His activates human B cells. Er-human blood mononuclear cells enriched for B cells were incubated either with media (black line), G28-8 (dotted line), or LH-HBcAgY132A-His (dashed line). Samples were analyzed 24 hours later for CD20 and CD40 expression using flow cytometry. Graph shows CD40 expression of gated CD20+B cells.

FIG. 5. IgG Antibody responses in macaques immunized and boosted with recombinant G28-8LH-HBcAgY132A-His vaccine (aCD180-HBVAg). Groups of rhesus macaques (Macaca mulatta) (N=3) were vaccinated subcutaneously with either: 1) G28-8LH-HBcAgY132A-His (ocCD180-HBVAg); 2) G28-8LH-HBcAgY132A-His (ocCD180-HBVAg) plus 1 mg long chain poly I:C; G28-8LH-HBcAgY132A-His (aCD180-HBVAg) plus CpGB; or 4) G28-8LH-HBcAgY132A-His encoding plasmid DNA (aCD180-HBVAg DNA) by delivering the DNA directly into cells of the skin using a gene gun. Animals were vaccinated on days 0, 30 and 81, and on days 0, 7, 14, 30 (time of first boost), 44, 62, 76, 90, 111 (time of second boost) 118, 125, 139 and 164 serum samples were obtained and evaluated for HBcAg-specific IgG antibody levels using ELISA. Endpoint titers are shown on a log scale.

FIG. 6. CD8 T cell responses in macaques immunized and boosted with recombinant G28-8LH-HBcAgY132A-His vaccine (HBcAg-CD180) detected by intracellular cytokine staining. Groups of rhesus macaques (Macaca mulatta) (N=3) were vaccinated as described in FIGS. 5 and 28 days after the second booster immunization, heparinized blood samples were obtained, from which PBMCs were isolated. PBMCs were incubated with HBcAg peptide pools. Cells were then stained with mAb specific for surface markers, followed by staining for intracellular cytokines. Fixed cells were then analyzed by multicolor flow cytometry. Shown for each of the four vaccinated groups are CD3+CD4+T cells (black) or CD3+CD8+T cells (gray) expressing either IFN-γ, TNF-α, IL-2 or CD107.

FIG. 7. HBcAg coupled to anti-CD180 (G28-8LH-HBcAgY132A-His) induces a strong IgG antibody response but free HBcAgY123A does not. Two groups of rhesus macaques (Macaca mulatta) (N=3) were immunized on day 0 and day 30 via the subcutaneous route with either 300 mg recombinant G28-8LH-HBcAgY132A-His in 1 ml (black) or an equivalent amount of HBcAgY132A (150 mg) in 1 ml (white) and bled day 0 before immunization and 14 days after the second immunization. Sera were then assessed for IgG antibodies to HBcAgY132A using ELISA using 96 well plates coated with 200 ng/well recombinant HBcAgY132A, serial dilutions of serum samples starting with a 1:1000 dilution and an HRP-anti-macaque IgG second step.

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise. “And” as used herein is interchangeably used with “or” unless expressly stated otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the invention can be used in combination, unless the context clearly dictates otherwise.

In a first aspect, the present invention provides compositions, comprising:
(a) a CD180 binding protein; and
(b) Hepatitis B virus core antigen (HBcAg) and/or Hepatitis B virus E antigen (HBeAg), or antigenic fragments or mutants thereof, attached to the CD180 binding protein.

The compositions of the invention can be used, for example, to induce prophylactic responses in individuals at risk of HBV infection, and therapeutic responses in already infected individuals and in immunodeficient individuals who do not respond well to standard vaccines. The present invention is highly significant because it provides a therapeutic vaccine for one of the major causes of cancer and liver disease in the world: hepatitis B virus (HBV). HBV infection is a serious global public health problem in both industrialized and developing nations. 10-30 million people worldwide become infected with HBV each year, and more than 2 billion people worldwide have been infected with HBV. Significantly, the ability of unvaccinated individuals to mount effective immune responses against HBV is correlated with age. Infants and young children are particularly at risk, as 90% of infants and up to 50% of young children infected with HBV ultimately develop chronic infections. About 400 million are chronically infected with HBV (CHB), and in the USA there are approximately 1.4 million CHB infected people⁵. In the USA the prevalence of HBV while dropping in children, has changed little in adults¹¹ such that the burden of chronic hepatitis B among adults remains large⁶; in some groups it is as high as 1%. An estimated 1 million people die each year from hepatitis and its complications, including about 5,000 people in the US. Of the 5000 persons in the United States who die each year from HBV related conditions, 300 die from fulminant hepatitis; 3-4000, from cirrhosis; and 600-1000, from primary hepatocellular carcinoma (HCC). In the US, approximately 400 health care workers are infected each year and are at risk from dying from HBV-related disease¹⁶.

The CD180 binding protein may be any molecule that binds directly to CD180 present in the surface of B cells, macrophages, or dendritic cells. In various non-limiting embodiments, the CD180 binding protein may be a peptide mimetic or an antibody.

In a particular embodiment, the CD180 binding protein an antibody or antibody fragment. As used herein, “antibody” includes reference to an immunoglobulin molecule immunologically reactive with human CD180 (preferably selective for CD180), and includes monoclonal antibodies. Various isotypes of antibodies exist, for example IgG1, IgG2, IgG3, IgG4, and other Ig, e.g., IgM, IgA, IgE isotypes. The term also includes genetically engineered forms such as chimeric antibodies (e.g., humanized murine antibodies) and heteroconjugate antibodies (e.g., bispecific antibodies), fully humanized antibodies, and human antibodies. As used throughout the application, the term “antibody” includes fragments with antigen-binding capability (e.g., Fab′, F(ab′)₂, Fab, Fv and rIgG. See also, Pierce Catalog and Handbook, 1994-1995 (Pierce Chemical Co., Rockford, IL). See also, e.g., Kuby, J., Immunology, 3^{rd} Ed., W.H. Freeman & Co., New York (1998). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al.. (1992) J Immunol 148:1547, Pack and Pluckthun (1992) Biochemistry 31:1579, Hollinger et al., 1993, supra, Gruber et al. (1994) J Immunol :5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301. Various antigen binding domain-fusion proteins are also disclosed, e.g., in US patent application Nos. 2003/0118592 and 2003/0133939, and are encompassed within the term “antibody” as used in this application.

An antibody immunologically reactive with human CD180 can be generated by recombinant methods such as selection of libraries of recombinant antibodies in phage or similar vectors, see, e.g., Huse et al., Science 246:1275-1281 (1989); Ward et al., Nature 341:544-546 (1989); and Vaughan et al., Nature Biotech. 14:309-314 (1996), or by immunizing an animal with the antigen or with DNA encoding the antigen.

In one embodiment, the CD180 binding protein comprises a single chain (sc) recombinant protein, wherein the sc recombinant protein comprises:
(i) a variable heavy (VH) chain region of an anti-CD180 antibody; and
(ii) a variable light (VL) chain region of an anti-CD180 antibody.

The VH and VL chain regions may be from an anti-human CD180 antibody, such as an anti-human CD180 monoclonal antibody. Exemplary commercially available CD180 anti-human monoclonal antibodies from which the VH and VL chains may be used include, but are not limited to, those sold by AbD Serotec (“MHR73”), BD Biosciences, Thermo Scientific, Sigma Aldrich, etc.), (“G28-8”), and LifeSpan (“200.1”). In one embodiment, the single chain recombinant protein does not include any other immunoglobulin domains (i.e.: a single chain variable fragment (scFv). In an alternative embodiment, the single chain recombinant protein further comprises: CH2 and CH3 domains from an immunoglobulin (Ig), such as a human Ig, or functional mutants thereof, wherein the CH2 and CH3 domains are located C-terminal to the VH and VL domains. The CH2 and CH3 domains may be from any immunoglobulin as deemed appropriate for an intended use of the composition, including but not limited to IgA1, IgA2, IgG1, IgG2, IgG3, IgG4, IgM, etc. In a particular embodiment, the sc recombinant protein comprises CH2 and CH3 domains from IgG1, such as human IgG1, or functional mutants thereof. In a particular embodiment, such “functional mutants” comprise CH2 and/or CH3 domains that have impaired binding to human or animal Fc receptor FcγRIIb and/or to human or animal complement proteins; the Fc domain of the recombinant molecules is an altered human IgG1 Fc domain with three amino acid changes (P238S, P331S, K322S) that reduce the binding of the molecule to Fc receptors and C1q. Other amino acid substitutions that can reduce binding of human IgG1 to various Fc receptors have been reported (J Biol Chem 276: 6591-6604) The ones that are most interesting includes E233, L234, L235, G236 deletion, P238, D265, N297, A327, and P329. Substitutions at these amino acids reduce binding to all FcγR (J Biol Chem 276: 6591-6604). Substitutions at D270, Q295, or A327 reduce binding to FcγRII and FcγRIIIA (J Biol Chem 276: 6591-6604). Substitutions at S239, E269, E293, Y296, V303, A327, K338, and D376 reduce binding to FcγRIIIA but not FcγRII (J Biol Chem 276: 6591-6604). A combination of two of more of these substitutions can be engineered in the Fc domains of human IgG1 to achieve the desired effects on inhibiting Fc-FcγR interaction between CD180 targeted vaccines and FcgR expressing cells. Similarly, modifying the glycosylation profile of human IgG1, for example, substitution of the N-linked glycosylation site at Asn-297 of human IgG1, eliminates N-linked glycosylation of human IgG1, thereby eliminating its binding to Fc receptors as well as complement fixation functions (John S. Axford (ed.), Glycobiology and Medicine, 27-43; 2005 Springer.

In these various embodiments of the compositions of the invention, the VL chain region may be located N-terminal to the VH chain region, or the VH chain region may be located N-terminal to the VL chain region, as disclosed in the examples that follow.

In one embodiment, the CD180-antibody comprises mAb G28-8, which is commercially available from a number of sources, (BD Biosciences, Thermo Scientific, Sigma Aldrich, etc.), a F(ab′)2 fragment of mAb G28-8, or a single chain recombinant protein having the VL and VH domains of G28-8, and optionally further comprising CH2 and CH3 domains from an immunoglobulin (Ig), such as a human Ig, or functional mutants thereof. The inventors have used a variety of such G28-8 based compositions in the examples herein to demonstrate activity of the compositions of the invention.

In another embodiment, the CD180 binding protein competes for binding to CD180 with monoclonal antibody G28-8. As used herein, competing CD180 binding proteins are those binding proteins that bind to about, substantially or essentially the same, or even the same, epitope as G28-8. Competing binding proteins, such as competing antibodies or derivatives thereof, include binding proteins with overlapping epitope specificities. Competing binding proteins are thus able to effectively compete with G28-8 antibody, such as the G28-8 antibody obtained from Thermo Scientific (the “reference antibody”) for binding to CD180. A binding protein that competes with the reference G28-8 antibody for binding to CD180 will be able to effectively or significantly reduce (i.e.: reduce by at least 10%; preferably by at least 20%, 30%, 40%, 50%, 60%, 70%, 80%, 90%, or more) reference G28-8 antibody binding to CD180, as evidenced by a reduction in bound label. In one embodiment, the reference G28-8 antibody is pre-mixed with varying amounts of the test binding proteins (e.g., 1:10, 1:100 or 1:1000) fora period of time prior to applying to a CD180 composition. In other embodiments, the reference G28-8 antibody and varying amounts of test binding proteins can simply be admixed during exposure to the CD180 composition. By using species or isotype secondary antibodies one will be able to detect only the bound reference G28-8 antibody, the binding of which will be reduced by the presence of a test binding protein that “competes” for binding. Detection of such binding events is well understood by those of skill in the art; examples are provided herein. For a further example, see Otipoby K L, Nagai Y, Shu G L, Miyake K, Clark E A. CD180 (RP105/Bgp95) workshop report. In Leukocyte Typing VII. White Cell Differentiation Antigens. Eds. D. Y. Mason et al., Oxford University Press, BC7, pp. 120-123, 2002.

As the identification of competing binding proteins is determined in comparison to the reference G28-8 antibody, it will be understood that actually determining the epitope to which the binding proteins bind is not in any way required in order to identify a competing binding proteins. However, epitope mapping can be performed using standard techniques, if desired.

The compositions of the invention comprise a Hepatitis B virus core antigen (HBcAg) and/or a Hepatitis B virus E antigen (HBeAg)), or antigenic fragments or mutants thereof (collectively referred to as HBcAg and/or HBeAg), attached to the CD180 binding protein. Thus, the compositions may comprise one or more HBeAgs and/or one or more HBeAgs. In all embodiment, one or more copies of HBcAgs and/or HBeAgs may be present at the N-terminus or the C-terminus of the single chain recombinant protein.

In one embodiment, the HBcAg and/or the HBeAg is capable of multimerizing (i.e.: forming dimers, trimers, tetramers, or other multimers). In a particular embodiment, the HBcAg and/or HBeAg are capable of dimerizing. In this embodiment, the HBV antigen forms the dimer in the composition. Recombinant proteins comprising HBeAg or wild-type HBcAg and dimeric anti-CD180 antibody or antibody fragments likely cannot be expressed efficiently. Thus, the inventors have used HBeAG and/or HBcAg that can form dimers, greatly enhancing efficient expression of the recombinant proteins of the invention. Furthermore, this design makes it (a) unnecessary to include a hinge region in the composition (as in a scAb) to create a dimer, and (b) acceptable to have a composition containing VL and VH only without any Fc domains, thereby reducing the risk of Fc-mediated depletion of the CD180-expressing antigen-presenting cells.

In one such embodiment, the HBcAg and/or HBeAg are capable of multimerization without the addition of an exogenous multimerization domain. For example, the HBV e-antigen (HBeAg), while being very closely related to HBcAg retains a propeptide that both blocks its interactions with HBcAg and enables it to form a loop structure and dimers. As a result, unlike HBcAg, HBeAg is capable of multimerizing, does not form capsids and is secreted from cells. In another embodiment, an HBcAg mutant having a single amino acid substitution of tyrosine to alanine at position 132 (HBcAgY132A) prevents the assembly of HBcAgY132A into higher order capsids. Instead, HBcAgY132A is expressed as a dimer. In a further embodiment, the HBcAg and/or the HBeAg comprise a multimerization domain, such as a dimerization domain. In these embodiments, the compositions do not form large, virus-like particles. Non-limiting examples of the numerous dimerization domains known to those of skill in the art and suitable for use in the present invention include, but are not limited to peptide helices containing at least one helix, or a structure formed by a helix, a coil and another helix, etc., coiled coil structures, dimerization domains within, for example, many cell surface signaling receptors, Fc regions or hinge regions of an antibody, leucine zippers, the STAT protein N terminal domain, FK506 binding protein, the LexA protein C-terminal domain, nuclear receptors, the FkpA N-terminal domain, orange carotenoid protein from A. maxima, M1 matrix protein from influenza, neuraminidase from influenza virus, E. coli fuculose aldolase; and the like. (see, e.g., O'Shea, Science. 254: 539 (1991), Barahmand-Pour et al., Curr. Top. Microbiol. Immunol. 211: 121-128 (1996); Klemm et al., Annu. Rev. Immunol. 16: 569-592 (1998); Klemm et al., Annu. Rev. Immunol. 16: 569-592 (1998); Ho et al., Nature. 382: 822-826 (1996); and Pomeranz et al., Biochem. 37: 965 (1998)). Further examples include residues 325 to 410 in the bovine papillomavirus E2 protein, (Dostatni, N., et al., EMBO J 7 (1988) 3807-3816; Haugen, T., et al. EMBO J 7 (1988) 4245-4253; McBride, A., et al., EMBO J 7 (1988) 533-539; McBride, A., et al., Proc Natl Acad Sci USA 86 (1989) 510-514),Type I deiodinase (D1): DFLVIYIEEAHASDGW (SEQ ID NO: 7) or ADFL--YI-EAH-DGW (SEQ ID NO: 63); HIV-1 Capsid Protein: QGPKEPFRDYVDRFYKTLRA (SEQ ID NO: 10); leucine zipper dimerization motif of yeast GCN4: HMKQL D VEEL S NYHL N VARL K VGER (SEQ ID NO: 22); leucine zipper in Escherichia coli transcriptional antiterminator protein; BgIG: GVTQLMREMLQLIKFQFSLNYQEESLSYQRLVT (SEQ ID NO: 23), EVSALEK (SEQ ID NO: 24) and/or KVSALKE (SEQ ID NO: 25).

In embodiments where the composition comprises HBcAg, the composition may comprise an HBcAg polypeptide at least 90% identical over its length to the amino acid sequence of SEQ ID NO:13, 14, 15, or 16.

| Full length HBcAg amino acid 1-179 | |
|---|---|
| SEQ ID NO: 13 | |
| 1 | MDIDPYKEFG ASVELLSFLP SDFFPSIRDL LDTASALYRE ALESPEHCSP HHTALRQAIL |
| 61 | CWGELMNLAT WVGSNLEDPA SRELVVSYVN VNMGLKIRQL LWFHISCLTF GRETVLEYLV |
| 121 | SFGVWIRTPP AYRPPNAPIL STLPETTVVR RRGRSPRRRT PSPRRRRSQS PRRRRSQSR |
| Full length HBcAg amino acid 1-179 with Y132A substitution | |
| SEQ ID NO: 14 | |
| 1 | MDIDPYKEFG ASVELLSFLP SDFFPSIRDL LDTASALYRE ALESPEHCSP HHTALRQAIL |
| 61 | CWGELMNLAT WVGSNLEDPA SRELVVSYVN VNMGLKIRQL LWFHISCLTF GRETVLEYLV |
| 121 | SFGVWIRTPP AARPPNAPIL STLPETTVVR RRGRSPRRRT PSPRRRRSQS PRRRRSQSR |
| Short HBcAg amino acid 1-149 | |
| SEQ ID NO: 15 | |
| 1 | MDIDPYKEFG ASVELLSFLP SDFFPSIRDL LDTASALYREA LESPEHCSP HHTALRQAIL |
| 61 | CWGELMNLAT WVGSNLEDPA SRELVVSYVN VNMGLKIRQL LWFHISCLTF GRETVLEYLV |
| 121 | SFGVWIRTPP AYRPPNAPIL STLPETTVV |
| Short HBcAg amino acid 1-149 with Y132A substitution | |
| SEQ ID NO: 16 | |
| 1 | MDIDPYKEFG ASVELLSFLP SDFFPSIRDL LDTASALYRE ALESPEHCSP HHTALRQAIL |
| 61 | CWGELMNLAT WVGSNLEDPA SRELVVSYVN VNMGLKIRQL LWFHISCLTF GRETVLEYLV |
| 121 | SFGVWIRTPP AARPPNAPIL STLPETTVV |

In various further embodiments, the composition may comprise an HBcAg polypeptide at least 91%, 92%, 93%, 94%, 95%, 96%, 97%, 98%, 99%, or 100% identical over its length to the amino acid sequence of SEQ ID NO:13, 14, 15, or 16. In various embodiments, additional HBcAg mutations may include (alone or in combination): F97L (J Virol. 2004 Sep;78(17):9538-43), V124W (J Virol. 2013 Mar;87(6):3208-16), substitution of residues that are phosphorylated in vivo, such as Ser87, Ser155, Ser162 and/or Ser170, to non-phosphorylatable residues (such as alanine or valie) (Biochem J. 2008 Nov 15;416(1):47-54; Biochem J. 2006 Sep 1;398(2):311-7), and/or substitution of one or more Cys residues (i.e.: Cys48, Cys61, Cys107, and/or Cys185) (to, for example, serine, alanine, or valine) which may increase dimerization (J Virol. 1992 Sep;66(9):5393-8).

In embodiments where the composition comprises HBeAg, the composition may comprise an HBeAg polypeptide at least 90% identical over its length to the amino acid sequence of SEQ ID NO:17.

(HBeAg)
SEQ ID NO: 17
S KLCLGWLWGM DIDPYKEFGA SVELLSFLPS DFFPSIRDLL
DTASALYREA LESPEHCSPH HTALRQAILC WGELMNLATW
VGSNLEDPAS RELVVSYVNV NMGLKIRQLL WFHISCLTFG
RETVLEYLVS FGVWIRTPPA YRPPNAPILS TLPETTVV

In various further embodiments, the composition may comprise an HBeAg polypeptide at least 91%, 92%, 93%, 94%, 95%, 96%, 97%, 98%, 99%, or 100% identical over its length to the amino acid sequence of SEQ ID NO: 17.

In all of these embodiments, the composition may further comprise an amino acid linker position between the CD180 binding protein and the HBcAg and/or HBeAg. The linker may be of any suitable length and amino acid composition, depending on the intended use. In one embodiment, the linker is between about 2-40 amino acids in length. In other embodiments, the linker may be between 10-30 or 15-25 amino acids in length. In another embodiment, the linker may be a linker rich in glycine and serine residues. In one specific embodiment, the linker may comprise the amino acid sequence

(SEQ ID NO: 4)
GGGGSGGGGSGGGGSGGGGS.

In various further embodiments, the composition may comprise or consist of a polypeptide at least 90% identical over its length to the amino acid sequence of SEQ ID NO:2, 3, 5, 6, 8, 9, 11, 12, 18, 19, 20, or 21.

| G28-8LH-HBcAgY132A-His Protein | |
|---|---|
| SEQ ID NO: 2 | |
| 1 | METPAQLLFL LLLWLPDTTG DIQMTQSPAS LSASVGETVT ITCRASEKIY SYLAWYQQKQ |
| 61 | GKSPQLLVYN AKTLAEGVPS RFSVSGSGTQ FSLRINSLQP EDFGTYYCQH HFGSPRTFGG |
| 121 | GTKLEIKDLG GGGSGGGGSG GGGSGGGGST GEVQLQQSGP ELVKPGASMK ISCKASGYSF |
| 181 | TGYTMNWVKQ SHGKTLEWIG LINPYNGVTS YNQKFKDKAT LTVDKSSSTA YMELLSLTSE |
| 241 | DSAIYYCARD YNYDYFDYWG QGTTLTVSSG GGGSGGGGSG GGGSGGGGSM DIDPYKEFGA |
| 301 | SVELLSFLPS DFFPSIRDLL DTASALYREA LESPEHCSPH HTALRQAILC WGELMNLATW |
| 361 | VGSNLEDPAS RELVVSYVNV NMGLKIRQLL WFHISCLTFG RETVLEYLVS FGVWIRTPPA |
| 421 | ARPPNAPILS TLPETTVV(HH HHHH) |
| 1-20: Leader | |
| Bold: G28-8VL | |
| Underlined: Gyl-Ser linkers | |
| Bold and underlined: G28-8VH | |
| Italicized and underlined: HBcAgY132A | |
| C-terminus: 6xHis | |
| G28-8LH-HBcAgY132A-His Mature Protein (leader sequence removed | |
| from SEQ ID NO: 2) | |
| SEQ ID NO: 3 | |
| 1 | DIQMTQSPAS LSASVGETVT ITCRASEKIY SYLAWYQQKQ GKSPQLLVYN AKTLAEGVPS |
| 61 | RFSVSGSGTQ FSLRINSLQP EDFGTYYCQH HFGSPRTFGG GTKLEIKDL G |
| | GGGSGGGGSG |
| 121 | GGGSGGGGST GEVQLQQSGP ELVKPGASMK ISCKASGYSF TGYTMNWVKQ SHGKTLEWIG |
| 181 | LINPYNGVTS YNQKFKDKAT LTVDKSSSTA YMELLSLTSE DSAIYYCARD YNYDYFDYWG |
| 241 | QGTTLTVSSG GGGSGGGGSG GGGSGGGGSM DIDPYKEFGA SVELLSFLPS DFFPSIRDLL |
| 301 | DTASALYREA LESPEHCSPH HTALRQAILC WGELMNLATW VGSNLEDPAS RELVVSYVNV |
| 361 | NMGLKIRQLL WFHISCLTFG RETVLEYLVS FGVWIRTPPA ARPPNAPILS |
| | TLPETTVV(HH |
| 421 | HHHH) |
| G28-8LH-HBcAg-His Protein | |
| SEQ ID NO: 5 | |
| 1 | METPAQLLFL LLLWLPDTTG DIQMTQSPAS LSASVGETVT ITCRASEKIY SYLAWYQQKQ |
| 61 | GKSPQLLVYN AKTLAEGVPS RFSVSGSGTQ FSLRINSLQP EDFGTYYCQH HFGSPRTFGG |
| 121 | GTKLEIKDLG GGGSGGGGSG GGGSGGGGST GEVQLQQSGP ELVKPGASMK ISCKASGYSF |
| 181 | TGYTMNWVKQ SHGKTLEWIG LINPYNGVTS YNQKFKDKAT LTVDKSSSTA YMELLSLTSE |
| 241 | DSAIYYCARD YNYDYFDYWG QGTTLTVSSG GGGSGGGGSG GGGSGGGGSM DIDPYKEFGA |
| 301 | SVELLSFLPS DFFPSIRDLL DTASALYREA LESPEHCSPH HTALRQAILC WGELMNLATW |
| 361 | VGSNLEDPAS RELVVSYVNV NMGLKIRQLL WFHISCLTFG RETVLEYLVS FGVWIRTPPA |
| 421 | YRPPNAPILS TLPETTVV(HH HHHH) |
| 1-20: Leader | |
| Bold: G28-8VL | |
| Underlined: Gyl-Ser linker | |
| Bold and underlined: G28-8VH | |
| Italicized and underlined: HBcAgY132A | |
| C-terminus: 6xHis | |
| G28-8LH-HBcAg-His Mature Protein (leader sequence removed | |
| from SEQ ID NO: 5) | |
| SEQ ID NO: 6 | |
| 1 | DIQMTQSPAS LSASVGETVT ITCRASEKIY SYLAWYQQKQ GKSPQLLVYN AKTLAEGVPS |
| 61 | RFSVSGSGTQ FSLRINSLQP EDFGTYYCQH HFGSPRTFGG GTKLEIKDLG GGGSGGGGSG |
| 121 | GGGSGGGGST GEVQLQQSGP ELVKPGASMK ISCKASGYSF TGYTMNWVKQ SHGKTLEWIG |
| 181 | LINPYNGVTS YNQKFKDKAT LTVDKSSSTA YMELLSLTSE DSAIYYCARD YNYDYFDYWG |
| 241 | QGTTLTVSSG GGGSGGGGSG GGGSGGGGSM DIDPYKEFGA SVELLSFLPS DFFPSIRDLL |
| 301 | DTASALYREA LESPEHCSPH HTALRQAILC WGELMNLATW VGSNLEDPAS RELVVSYVNV |
| 361 | NMGLKIRQLL WFHISCLTFG RETVLEYLVS FGVWIRTPPA YRPPNAPILSTLPETTVV(HH |
| 421 | HHHH) |
| G28-8LH-HBcAgY132AFL-His Protein | |
| SEQ ID NO: 8 | |
| 1 | METPAQLLFL LLLWLPDTTG DIQMTQSPAS LSASVGETVT ITCRASEKIY SYLAWYQQKQ |
| 61 | GKSPQLLVYN AKTLAEGVPS RFSVSGSGTQ FSLRINSLQP EDFGTYYCQH HFGSPRTFGG |
| 121 | GTKLEIKDLG GGGSGGGGSG GGGSGGGGST GEVQLQQSGP ELVKPGASMK ISCKASGYSF |
| 181 | TGYTMNWVKQ SHGKTLEWIG LINPYNGVTS YNQKFKDKAT LTVDKSSSTA YMELLSLTSE |
| 241 | DSAIYYCARD YNYDYFDYWG QGTTLTVSSG GGGSGGGGSG GGGSGGGGSM DIDPYKEFGA |
| 301 | SVELLSFLPS DFFPSIRDLL DTASALYREA LESPEHCSPH HTALRQAILC WGELMNLATW |
| 361 | VGSNLEDPAS RELVVSYVNV NMGLKIRQLL WFHISCLTFG RETVLEYLVS FGVWIRTPPA |
| 421 | ARPPNAPILS TLPETTVVRR RGRSPRRRTP SPRRRRSQSP RRRRSQSR(HH HHHH) |
| 1-20: Leader | |
| Bold: G28-8VL | |
| Underlined: Gyl-Ser linker | |
| Bold and underlined: G28-8VH | |
| Italicized and underlined: Full length HBcAg protein with Y132A | |
| substitution | |
| C-terminus: 6xHis | |
| G28-8LH-HBcAgY132AFL-His Mature Protein (leader sequence removed | |
| from SEQ ID NO: 8) | |
| SEQ ID NO: 9 | |
| 1 | DIQMTQSPAS LSASVGETVT ITCRASEKIY SYLAWYQQKQ GKSPQLLVYN AKTLAEGVPS |
| 61 | RFSVSGSGTQ FSLRINSLQP EDFGTYYCQH HFGSPRTFGG GTKLEIKDLG GGGSGGGGSG |
| 121 | GGGSGGGGST GEVQLQQSGP ELVKPGASMK ISCKASGYSF TGYTMNWVKQ SHGKTLEWIG |
| 181 | LINPYNGVTS YNQKFKDKAT LTVDKSSSTA YMELLSLTSE DSAIYYCARD YNYDYFDYWG |
| 241 | QGTTLTVSSG GGGSGGGGSG GGGSGGGGSM DIDPYKEFGA SVELLSFLPS DFFPSIRDLL |
| 301 | DTASALYREA LESPEHCSPH HTALRQAILC WGELMNLATW VGSNLEDPAS RELVVSYVNV |
| 361 | NMGLKIRQLL WFHISCLTFG RETVLEYLVS FGVWIRTPPA ARPPNAPILS TLPETTVVRR |
| 421 | RGRSPRRRTP SPRRRRSQSP RRRRSQSR(HH HHHH) |
| G28-8LH-HBcAgFL-His Protein | |
| SEQ ID NO: 11 | |
| 1 | METPAQLLFL LLLWLPDTTG DIQMTQSPAS LSASVGETVT ITCRASEKIY SYLAWYQQKQ |
| 61 | GKSPQLLVYN AKTLAEGVPS RFSVSGSGTQ FSLRINSLQP EDFGTYYCQH HFGSPRTFGG |
| 121 | GTKLEIKDLG GGGSGGGGSG GGGSGGGGST GEVQLQQSGP ELVKPGASMK ISCKASGYSF |
| 181 | TGYTMNWVKQ SHGKTLEWIG LINPYNGVTS YNQKFKDKAT LTVDKSSSTA YMELLSLTSE |
| 241 | DSAIYYCARD YNYDYFDYWG QGTTLTVSSG GGGSGGGGSG GGGSGGGGSM DIDPYKEFGA |
| 301 | SVELLSFLPS DFFPSIRDLL DTASALYREA LESPEHCSPH HTALRQAILC WGELMNLATW |
| 361 | VGSNLEDPAS RELVVSYVNV NMGLKIRQLL WFHISCLTFG RETVLEYLVS FGVWIRTPPA |
| 421 | YRPPNAPILS TLPETTVVRR RGRSPRRRTP SPRRRRSQSP RRRRSQSR(HH HHHH) |
| 1-20: Leader | |
| Bold: G28-8VL | |
| Underlined: Gyl-Ser linker | |
| Bold and underlined: G28-8VH | |
| Italicized and underlined: Full length HBcAg protein | |
| C-terminus: 6xHis | |
| G28-8LH-HBcAgFL-His Mature Protein (leader sequence removed | |
| from SEQ ID NO: 11) | |
| SEQ ID NO: 12 | |
| 1 | DIQMTQSPAS LSASVGETVT ITCRASEKIY SYLAWYQQKQ GKSPQLLVYN AKTLAEGVPS |
| 61 | RFSVSGSGTQ FSLRINSLQP EDFGTYYCQH HFGSPRTFGG GTKLEIKDLG GGGSGGGGSG |
| 121 | GGGSGGGGST GEVQLQQSGP ELVKPGASMK ISCKASGYSF TGYTMNWVKQ SHGKTLEWIG |
| 181 | LINPYNGVTS YNQKFKDKAT LTVDKSSSTA YMELLSLTSE DSAIYYCARD YNYDYFDYWG |
| 241 | QGTTLTVSSG GGGSGGGGSG GGGSGGGGSM DIDPYKEFGA SVELLSFLPS DFFPSIRDLL |
| 301 | DTASALYREA LESPEHCSPH HTALRQAILC WGELMNLATW VGSNLEDPAS RELVVSYVNV |
| 361 | NMGLKIRQLL WFHISCLTFG RETVLEYLVS FGVWIRTPPA YRPPNAPILS TLPETTVVRR |
| 421 | RGRSPRRRTP SPRRRRSQSP RRRRSQSR(HH HHHH) |

G28-8scFVLH-HBeAg
SEQ ID NO: 18
METPAQLLFL LLLWLPDTTG DIQMTQSPAS LSASVGETVT ITCRASEKIY
SYLAWYQQKQ GKSPQLLVYN AKTLAEGVPS RFSVSGSGTQ FSLRINSLQP
EDFGTYYCQH HFGSPRTFGG GTKLEIKDLG GGGSGGGGSG GGGSGGGGST
GEVQLQQSGP ELVKPGASMK ISCKASGYSF TGYTMNWVKQ SHGKTLEWIG
LINPYNGVTS YNQKFKDKAT LTVDKSSSTA YMELLSLTSE DSAIYYCARD
YNYDYFDYWG QGTTLTVSSG GGGSGGGGSG GGGSGGGGSS KLCLGWLWGM
DIDPYKEFGA SVELLSFLPS DFFPSIRDLL DTASALYREA LESPEHCSPH
HTALRQAILC WGELMNLATW VGSNLEDPAS RELVVSYVNV NMGLKIRQLL
WFHISCLTFG RETVLEYLVS FGVWIRTPPA YRPPNAPILS TLPETTVV(HH
HHHH)
1-20: Leader
Bold: G28-8VL
Underlined: Gyl-Ser linkers
Bold and underlined: G28-8VH
Italicized and underlined: HBeAg
C-terminus: 6xHis
Mature G28-8scFVLH-HBeAg protein (leader sequence removed
from SEQ ID NO: 18)
SEQ ID NO: 19
DIQMTQSPAS LSASVGETVT ITCRASEKIY SYLAWYQQKQ GKSPQLLVYN
AKTLAEGVPS RFSVSGSGTQ FSLRINSLQP EDFGTYYCQH HFGSPRTFGG
GTKLEIKDLG GGGSGGGGSG GGGSGGGGST GEVQLQQSGP ELVKPGASMK
ISCKASGYSF TGYTMNWVKQ SHGKTLEWIG LINPYNGVTS YNQKFKDKAT
LTVDKSSSTA YMELLSLTSE DSAIYYCARD YNYDYFDYWG QGTTLTVSSG
GGGSGGGGSG GGGSGGGGSS KLCLGWLWGM DIDPYKEFGA SVELLSFLPS
DFFPSIRDLL DTASALYREA LESPEHCSPH HTALRQAILC WGELMNLATW
VGSNLEDPAS RELVVSYVNV NMGLKIRQLL WFHISCLTFG RETVLEYLVS
FGVWIRTPPA YRPPNAPILS TLPETTVV(HH HHHH)
HBeAg-G28-8scFVLH
SEQ ID NO: 20
MQLFPLCLII SCSCPTVQAS KLCLGWLWGM DIDPYKEFGA SVELLSFLPS
DFFPSIRDLL DTASALYREA LESPEHCSPH HTALRQAILC WGELMNLATW
VGSNLEDPAS RELVVSYVNV NHGLKIRQLL WFHISCLTFG RETVLEYLVS
FGVWIRTPPA YRPPNAPILS TLPETTVVGG GGSGGGGSGG GGSGGGGSDI
QMTQSPASLS ASVGETVTIT CRASEKIYSY LAWYQQKQGK SPQLLVYNAK
TLAEGVPSRF SVSGSGTQFS LRINSLQPED FGTYYCQHHF GSPRTFGGGT
KLEIKDLGGG GSGGGGSGGG GSGGGGSTGE VQLQQSGPEL VKPGASMKIS
CKASGYSFTG YTMNWVKQSH GKTLEWIGLI NPYNGVTSYN QKFKDKATLT
VDKSSSTAYM ELLSLTSEDS AIYYCARDYN YDYFDYWGQG TTLTVSS(HHH
HHH)
1-19: Leader
Bold: G28-8VL
Underlined: Gyl-Ser linkers
Bold and underlined: G28-8VH
Italicized and underlined: HBeAg
C-terminus: 6xHis
Mature HBeAg-G28-8scFVLH protein
SEQ ID NO: 21
SKLCLGWLWG MDIDPYKEFG ASVELLSFLP SDFFPSIRDL LDTASALYRE
ALESPEHCSP HHTALRQAIL CWGELMNLAT WVGSNLEDPA SRELVVSYVN
VNMGLKIRQL LWFHISCLTF GRETVLEYLV SFGVWIRTPP AYRPPNAPIL
STLPETTVVG GGGSGGGGSG GGGSGGGGSD IQMTQSPASL SASVGETVTI
TCRASEKIYS YLAWYQQKQG KSPQLLVYNA KTLAEGVPSR FSVSGSGTQF
SLRINSLQPE DFGTYYCQHH FGSPRTFGGG TKLEIKDLGG GGSGGGGSGG
GGSGGGGSTG EVQLQQSGPE LVKPGASMKI SCKASGYSFT GYTMNWVKQS
HGKTLEWIGL INPYNGVTSY NQKFKDKATL TVDKSSSTAY MELLSLTSED
SAIYYCARDY NYDYFDYWGQ GTTLTVSS(HH HHHH)
Purple: Leader
Brown: G28-8VL
Red: Gyl-Ser linker
Black: G28-8VH
Blue: Full length HBeAg protein
Bright green: 6xHis

The compositions of any embodiment or combination of embodiments of the invention may be provided as a stand-alone composition, or may be provided as part of a molecular scaffold. In various embodiments, the composition may be attached to molecular scaffold. Any suitable scaffold can be used, including but not limited to a VNAR single domain antibody (shark variable new antigen receptor), a lamprey variable lymphocyte receptor, a Im 7(colicin immunity 7 protein), an anticalin (lipocalin transport proteins), an FN3 (fibronectin 3) monobody, a DARPin (designed ankyrin repeat proteins), an affibody (Z domain of protein A), etc., with CD180-binding polypeptide loops.

In another embodiment, the composition of any embodiment or combination of embodiments of the invention further comprises an adjuvant. While adjuvant is not required to induce rapid activation of HBcAg and/or HBeAg-specific B cells, addition of adjuvant to the compositions can result in additional enhancement of the immune response when the compositions are used in the methods of the invention. Any suitable adjuvant can be used, including but not limited to inorganic compounds (aluminum hydroxide, aluminum phosphate, calcium phosphate hydroxide, beryllium, etc.), mineral oil, detergents, cytokines, toll-like receptor agonists, Freund's complete adjuvant, Freund's incomplete adjuvant, squalene, etc. In a preferred embodiment, the adjuvant comprises or consists of a toll-like receptor 4 (TLR4) agonist, a toll-like receptor 7 (TLR7) agonist, a toll-like receptor 8 (TLR8) agonist, a toll-like receptor 9 (TLR9) agonist, alum-containing adjuvant, monophosphoryl lipid A, oil-in-water emulsion, and α-tocopherol, squalene and polysorbate 80 in an oil-in-water emulsion. The adjuvant may be present in the composition as an unlinked component or a linked component, depending on the adjuvant used.

In another embodiment, the compositions of the invention can be modified to extend half-life, such as by attaching at least one molecule to the composition for extending serum half-life, including but not limited to a polyethlyene glycol (PEG) group, serum albumin, transferrin, transferrin receptor or the transferrin-binding portion thereof, or combinations thereof. As used herein, the word “attached” refers to a covalently or noncovalently conjugated substance. The conjugation may be by genetic engineering or by chemical means.

The compositions of the present invention may be stored in any suitable buffer.

In a second aspect, the present invention provides isolated nucleic acids encoding the composition of any embodiment of the first aspect of the invention. The isolated nucleic acid sequence may comprise RNA or DNA. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. In various non-limiting embodiments, the isolated nucleic acids encode the polypeptide of any one of SEQ ID NOS: 2, 3, 5, 6, 8, 9, 11, or 12. In other embodiments, the isolated nucleic acids comprise or consist of the nucleotide sequence of SEQ ID NO:1, 4, 7, or 10.

In a third aspect, the present invention provides nucleic acid vectors comprising the isolated nucleic acid of the second aspect of the invention. “Recombinant expression vector” includes vectors that operatively link a nucleic acid coding region or gene to any promoter capable of effecting expression of the gene product. The promoter sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The construction of expression vectors for use in transfecting prokaryotic cells is also well known in the art, and thus can be accomplished via standard techniques. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, TX). The expression vector must be replicable in the host organisms either as an episome or by integration into host chromosomal DNA. In a preferred embodiment, the expression vector comprises a plasmid. However, the invention is intended to include other expression vectors that serve equivalent functions, such as viral vectors.

The nucleic acids and vectors of the invention can be used not only for production of large quantities of the compositions of the invention, but also for use as a nucleic acid (such as a DNA) vaccine administered by gene gun or other methods.

In a fourth aspect, the present invention provides recombinant host cells comprising the nucleic acid vector of the third aspect of the invention. The host cells can be either prokaryotic or eukaryotic. The cells can be transiently or stably transfected. Such transfection of expression vectors into prokaryotic and eukaryotic cells (including but not limited to Chinese hamster ovary (CHO) cells) can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2^{nd} Ed. (R. I. Freshney. 1987. Liss, Inc. New York, NY).

The recombinant host cells can be used, for example in methods for producing antibody (when the binding protein is an antibody), comprising:
(a) culturing the recombinant host cell of the invention under conditions suitable for expression of the nucleic-acid encoded antibody composition; and
(b) isolating the antibody composition from the cultured cells.

Suitable conditions for expression of the nucleic-acid encoded antibody composition can be determined by those of skill in the art based on the teachings herein, the specific host cells and vectors used, and the general knowledge of those of skill in the art.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes disclosed herein. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes disclosed herein.

In a fifth aspect, the present invention provides pharmaceutical compositions, comprising:
(a) the composition, isolated nucleic acid, or recombinant expression vector of any embodiment or combination of embodiments disclosed herein; and
(b) a pharmaceutically acceptable carrier. In this embodiment, the compositions of the invention are present in a pharmaceutical formulation. In this embodiment, the compositions are combined with a pharmaceutically acceptable carrier. Suitable acids which are capable of forming such salts include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid and the like. Suitable bases capable of forming such salts include inorganic bases such as sodium hydroxide, ammonium hydroxide, potassium hydroxide and the like; and organic bases such as mono-, di- and tri-alkyl and aryl amines (e.g., triethylamine, diisopropyl amine, methyl amine, dimethyl amine and the like) and optionally substituted ethanol-amines (e.g., ethanolamine, diethanolamine and the like).

The pharmaceutical composition may comprise in addition to the composition of the invention (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The pharmaceutical compositions of the invention may be made up in any suitable formulation, preferably in formulations suitable for administration by injection. Such pharmaceutical compositions can be used, for example, in methods of use as vaccines, prophylactics, or therapeutics.

The pharmaceutical compositions may contain any other components as deemed appropriate for a given use, such as additional therapeutics or vaccine components. In one embodiment, the pharmaceutical compositions further comprise toll-like receptor 4 (TLR4) agonist, a toll-like receptor 7 (TLR7) agonist, a toll-like receptor 8 (TLR8) agonist, a toll-like receptor 9 (TLR9) agonist, alum-containing adjuvant, monophosphoryl lipid A, oil-in-water emulsion, and α-tocopherol, squalene and polysorbate 80 in an oil-in-water emulsion.

In a sixth aspect, the present invention provides methods for treating or limiting development of an HBV infection or a hepatitis-B virus (HBV)-related disorder, comprising administering to an individual in need thereof an amount effective to treat or limit development of the disorder of the composition, isolated nucleic acid, recombinant expression vector, or pharmaceutical composition, or a pharmaceutical salt thereof, of any embodiment or combination of embodiments of the present invention. In one embodiment, the compositions are used prophylactically as vaccines to limit development of HBV infection disease/severity of infectious disease, such as in individuals that have not been exposed to an infectious agent but are at risk of such exposure. In other embodiments, the methods can be used therapeutically to treat people exposed to or chronically infected with HBV.

The methods of the invention target antigen to CD180, a surface protein expressed on B cells, macrophages, and dendritic cells, that to produce antigen-specific IgG in the absence of T cell costimulation (such as CD40 deficiency) or the complete absence of T cells (such as TCR β/δ deficiency). Thus, the methods can be used in any therapeutic or prophylactic treatment for HBV infection or vaccination. This approach also finds use, for example, for neonates, the elderly, and the immunodeficient, both in specifically targeting cellular populations enriched in underdeveloped or otherwise deficient immune systems and by improving responses to antigens that require linked recognition (carbohydrate epitopes, etc.).

As used herein, “treat” or “treating” means accomplishing one or more of the following in an individual that already has a disorder or has already been exposed to a disorder-causing substance/pathogen: (a) reducing the severity of the disorder; (b) limiting or preventing development of symptoms characteristic of the disorder(s) being treated (ex: immune deficiencies in cancer patients or other patients) undergoing chemotherapy and/or radiation therapy); (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, “limiting” or “limiting development of” means accomplishing one or more of the following in an individual that does not have the disorder to be limited: (a) preventing the disorder; (b) reducing the severity of the disorder; and (c) limiting or preventing development of symptoms characteristic of the disorder.

As used herein, an “amount effective” refers to an amount of the composition that is effective for treating and/or limiting the relevant disorder.

While the methods of the invention do not require use of an adjuvant, the methods may further comprise administering an adjuvant for possible additional enhancement of the immune response Any suitable adjuvant can be used, including but not limited to toll-like receptor 4 (TLR4) agonist, a toll-like receptor 7 (TLR7) agonist, a toll-like receptor 8 (TLR8) agonist, a toll-like receptor 9 (TLR9) agonist, alum-containing adjuvant, monophosphoryl lipid A, oil-in-water emulsion, and α-tocopherol, squalene and polysorbate 80 in an oil-in-water emulsion.

The individual may be any suitable individual, including but not limited to mammals. Preferably the individual is a human. In one embodiment, the individual has a T-cell deficiency and/or a defect in co-stimulation between B cells and T cells, or is immuno-compromised by chronic infections or from acute or chronic taking of immunosuppressive drugs for treatment of autoimmune diseases, or other inflammatory disease. In another embodiment, the individual is less than one month old or is elderly (i.e.: at least 65 years old).

In various other embodiments, the individual has a hepatitis B-related disease, such as hepatitis, hepatitis-related disease, fulminant hepatitis, cirrhosis, and/or hepatocellular carcinoma, and the methods are used to treat the a hepatitis B-related disease, such as hepatitis, hepatitis-related disease, fulminant hepatitis, cirrhosis, and/or hepatocellular carcinoma.

### EXAMPLE 1

### Generation and Characterization of G28-8LH-HBcAgY13A-His Recombinant Vaccine Molecule

G28-8 (anti-human CD180) scFv-HBcAg recombinant vaccine molecules. The inventors have demonstrated that for the specific anti-CD180 antibody, G28-8, a single chain antibody (scAb) in the form of VLVH-human IgG1 Fc retains both the efficient binding as well as the biological properties of its parent G28-8 IgG. The G28-8LH single chain Fv (scFv) was used to create G28-8scFv-HBcAg recombinant vaccine constructs. scFv generated from other anti-CD180 antibodies should retain the antibody characteristics in either the VLVH, or the VHVL configuration.

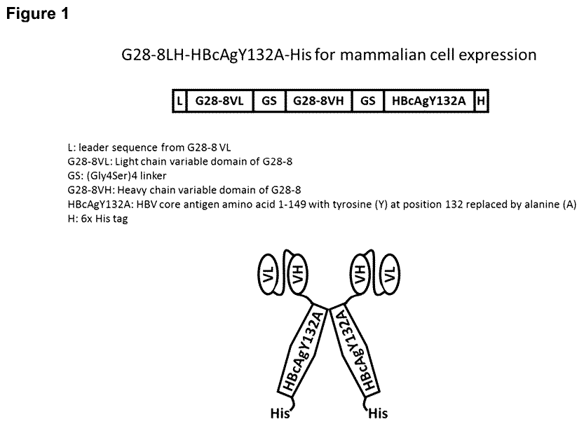

HBV capsid proteins that do not self-assemble can be attached to anti-CD180 scFv and expressed. Normally HBV capsid proteins (HBcAgs) self-assemble around the pre-genomic HBV RNA and the viral reverse transcriptase. The assembly domain of the HBcAg plays a key role in the assembly of the capsid and its stability. The tyrosine residue Y132 is required to stabilize the interactions between HBV core Ag dimers, which in turn is required for icosahedral capsids to form. HBcAg proteins with a single mutation at Y132 to alanine (Y132A) have defective capsid assembly.^{38,39} The HBcAgY132A mutant is a stable dimer but unlike wildtype HBcAg, does not assemble into capsids even at high salt solution (1 M NaCl). Because HBcAg-Y132A proteins form dimers and crosslinking of CD180 is required for optimal signaling, we proposed that anti-CD180 scFv-HBcAgY132A proteins may form dimers. Because scFv G28-8LH has shown to retain the binding and B cell stimulatory activity, it was used to construct the recombinant vaccine molecule consisting of HBcAgY132A. The dimeric nature of anti-CD180 scFv in such G28-8LH-HBcAgY132A protein was in turn be able to functionally engage CD180 molecules on antigen-presenting to activate them, e.g., activation of B cells. We designed a DNA sequence encoding a protein comprised of the VL and VH domains of G28-8 (anti-CD180), a Glycine-Serine linker, the HBcAgY132A mutant protein, and a His tag at the C-terminal for affinity purification of the recombinant vaccine molecule (FIG. 1, SEQ ID NO:1).

| G28-8LH-HBcAgY132A-His | |
|---|---|
| SEQ ID NO: 1 | |
| 1 | GCGAAGCTTT GAGCCACCAT GGAAACCCCA GCGCAGCTTC TCTTCCTCCT GCTACTCTGG |
| 61 | CTCCCAGATA CCACCGGTGA CATCCAGATG ACTCAGTCTC CAGCCTCCCT ATCTGCATCT |
| 121 | GTGGGAGAAA CTGTCACCAT CACATGTCGA GCAAGTGAGA AGATTTACAG TTATTTAGCA |
| 181 | TGGTATCAGC AGAAACAGGG AAAATCTCCT CAGCTCCTGG TCTATAACGC AAAAACCTTA |
| 241 | GCAGAAGGTG TGCCATCAAG GTTCAGTGTC AGTGGATCAG GCACACAGTT TTCTCTGAGG |
| 301 | ATCAACAGCC TGCAGCCTGA AGATTTTGGG ACTTATTACT GTCAACATCA TTTTGGTTCT |
| 361 | CCTCGGACGT TCGGTGGAGG CACCAAACTG GAAATCAAAG ATCTCGGAGG AGGTGGCTCA |
| 421 | GGTGGTGGAG GATCTGGAGG AGGTGGGAGT GGTGGAGGTG GTTCTACCGG TGAGGTCCAG |
| 481 | CTGCAACAGT CTGGACCTGA ACTGGTGAAG CCTGGAGCTT CAATGAAGAT ATCCTGCAAG |
| 541 | GCTTCTGGTT ACTCATTCAC TGGCTACACC ATGAACTGGG TGAAGCAGAG CCATGGAAAG |
| 601 | ACCCTTGAAT GGATTGGACT TATTAATCCT TACAATGGTG TTACTAGCTA CAACCAGAAG |
| 661 | TTCAAGGACA AGGCCACATT AACTGTAGAC AAGTCATCCA GCACAGCCTA CATGGAACTC |
| 721 | CTCAGTCTGA CATCTGAGGA CTCTGCAATC TATTACTGTG CAAGAGACTA TAATTACGAC |
| 781 | TACTTTGACT ACTGGGGCCA AGGCACCACT CTCACAGTCT CCTCAGGAGG AGGTGGCTCA |
| 841 | GGTGGTGGAG GATCTGGAGG AGGTGGGAGT GGTGGAGGTG GTTCTATGGA CATTGACCCG |
| 901 | TATAAAGAAT TTGGAGCTTC TGTGGAGTTA CTCTCTTTTT TGCCTTCTGA CTTCTTTCCT |
| 961 | TCTATTCGAG ATCTCCTCGA CACCGCCTCT GCTCTGTATC GGGAGGCCTT AGAGTCTCCG |
| 1021 | GAACATTGTT CACCTCACCA TACAGCACTC AGGCAAGCTA TTCTGTGTTG GGGTGAGTTG |
| 1081 | ATGAATCTGG CCACCTGGGT GGGAAGTAAT TTGGAAGACC CAGCATCCAG GGAATTAGTA |
| 1141 | GTCAGCTATG TCAATGTTAA TATGGGCCTA AAAATTAGAC AACTATTGTG GTTTCACATT |
| 1201 | TCCTGCCTTA CTTTTGGAAG AGAAACTGTC CTTGAGTATT TGGTGTCTTT TGGAGTGTGG |
| 1261 | ATTCGCACTC CTCCCGCTGC CAGACCACCA AATGCCCCTA TCTTATCAAC ACTTCCGGAA |
| 1321 | ACTACTGTTG TTCACCACCA TCATCATCAT TGATAAGGAT CCGCG |

5′ end HindIII and 3' end BamHI sites for
directional cloning into appropriate expression
vector
Kozak consensus, GCCACC, right before 5′ ATG start
codon
One 5′ in frame stop codon after 5′ end HindIII
site
Two in frame stop codons before 3′ end BamHI site

The native leader sequence from the VL domain of G28-8 was included to facilitate secretion of the recombinant vaccine molecules from the host CHO cells (SEQ ID NO: 1, SEQ ID NO:2).

| G28-8LH-HBcAgY132A-His Protein | |
|---|---|
| SEQ ID NO: 2 | |
| 1 | METPAQLLFL LLLWLPDTTG DIQMTQSPAS LSASVGETVT ITCRASEKIY SYLAWYQQKQ |
| 61 | GKSPQLLVYN AKTLAEGVPS RFSVSGSGTQ FSLRINSLQP EDFGTYYCQH HFGSPRTFGG |
| 121 | GTKLEIKDLG GGGSGGGGSG GGGSGGGGST GEVQLQQSGP ELVKPGASMK ISCKASGYSF |
| 181 | TGYTMNWVKQ SHGKTLEWIG LINPYNGVTS YNQKFKDKAT LTVDKSSSTA YMELLSLTSE |
| 241 | DSAIYYCARD YNYDYFDYWG QGTTLTVSSG GGGSGGGGSG GGGSGGGGSM DIDPYKEFGA |
| 301 | SVELLSFLPS DFFPSIRDLL DTASALYREA LESPEHCSPH HTALRQAILC WGELMNLATW |
| 361 | VGSNLEDPAS RELVVSYVNV NMGLKIRQLL WFHISCLTFG RETVLEYLVS FGVWIRTPPA |
| 421 | ARPPNAPILS TLPETTVV(HH HHHH) |

1-20: Leader
Bold: G28-8VL
Underlined: Gyl-Ser linkers
Bold and underlined: G28-8VH
Italicized and underlined: HBcAgY132A
C-terminus: 6xHis

The predicted mature polypeptide sequence after the cleavage of the leader sequence is given in SEQ ID NO: 3. The HBcAgY132A sequence in this vaccine construct consists of amino acid residues 1 to 149 of HBcAg without the C-terminal arginine-rich resides of 150-183.

| G28-8LH-HBcAgY132A-His Mature Protein (leader | |
|---|---|
| sequence removed from SEQ ID NO: 2) | |
| SEQ ID NO: 3 | |
| 1 | DIQMTQSPAS LSASVGETVT ITCRASEKIY SYLAWYQQKQ GKSPQLLVYN AKTLAEGVPS |
| 61 | RFSVSGSGTQ FSLRINSLQP EDFGTYYCQH HFGSPRTFGG GTKLEIKDLG GGGSGGGGSG |
| 121 | GGGSGGGGST GEVQLQQSGP ELVKPGASMK ISCKASGYSF TGYTMNWVKQ SHGKTLEWIG |
| 181 | LINPYNGVTS YNQKFKDKAT LTVDKSSSTA YMELLSLTSE DSAIYYCARD YNYDYFDYWG |
| 241 | QGTTLTVSSG GGGSGGGGSG GGGSGGGGSM DIDPYKEFGA SVELLSFLPS DFFPSIRDLL |
| 301 | DTASALYREA LESPEHCSPH HTALRQAILC WGELMNLATW VGSNLEDPAS RELVVSYVNV |
| 361 | NMGLKIRQLL WFHISCLTFG RETVLEYLVS FGVWIRTPPA ARPPNAPILS TLPETTVV(HH |
| 421 | HHHH) |

Production of recombinant the G28-8LH-HBcAgY132A-His protein. Complementary DNAs (cDNAs) encoding the G28-8LH-HBcAgY132A-His recombinant proteins (FIG. 1, SEQ ID NO:1) were cloned into the mammalian expression vector pTT5 that harbors a CMV promoter to drive protein expression. Transient transfection of these plasmids into Chinese hamster ovary (CHO) cells was done using Lipofectamine reagents (Invitrogen Carlsbad, CA) or polyethyleninmine (PEI). Small-scale transfection optimization using 5%, 20% and 80% ratios of expression plasmid in the lipofection reagent was conducted to identify to optimal plasmid to lipofection reagent ratios for larger scale expression. Nickel affinity chromatography, e.g., using the HisPurNi-NTA resin (Thermo Fisher Scientific Inc., Rockford IL), was used to purify the recombinant proteins. The cDNA sequences for the G28-8LH-HBcAgY132A-His protein predicts a polypeptide size of ˜45 kDa.

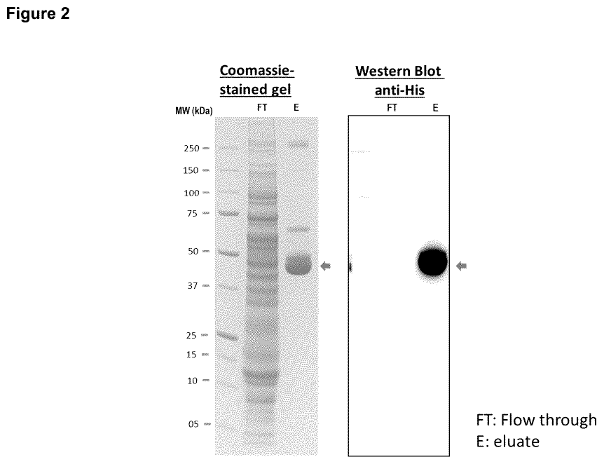

FIG. 2 shows the results from a 2-liter expression run. The plasmid encoding the G28-8LH-HBcAg-Y132A-His protein was transiently expressed in CHO cells for 8 days. Culture supernatants (˜2 liters) were collected and cellular debris was removed by centrifugation. Clarified culture supernatants were then loaded on to a column containing HisPurNi-NTA resin. After washing the column with the wash buffer (50 mM phosphate buffer, pH 7.0, 300 mM NaCl, 1 mM imidazole), bound recombinant protein was eluted with the elution buffer (50 mM phosphate buffer, pH 7.0, 300 mM NaCl, 150 mM imidazole). Protein containing fractions as monitored by absorbance at 280 nM were collected, pooled, and dialyzed against phosphate-buffer saline at pH 7.0. Purified G28-8LH-HBcAgY312A-His and unbound flow through materials from HisPurNi-NTA chromatography was analyzed on SDS-PAGE (4-15% gradient under reducing conditions) stained with Coomassie blue. FIG. 2, left panel shows a major protein band migrating at the predicted MW of ˜45 kDa, suggesting that G28-8LH-HBcAgY132A-His protein was in fact expressed by CHO cells as an intact protein and secreted into the culture supernatants. A duplicate gel was then transferred onto a nylon membrane and immuno-blotted with an anti-6x-His antibody. Intense anti-6xHis signals were only observed at ˜45 kDa (FIG. 2, right panel), at the identical MW G28-8LH-HBcAgY132A-His migrated to on the Coomassie blue stained gel (FIG. 2, left panel).

### EXAMPLE 2

### Characterization of G28-8LH-HBcAgY132A-His

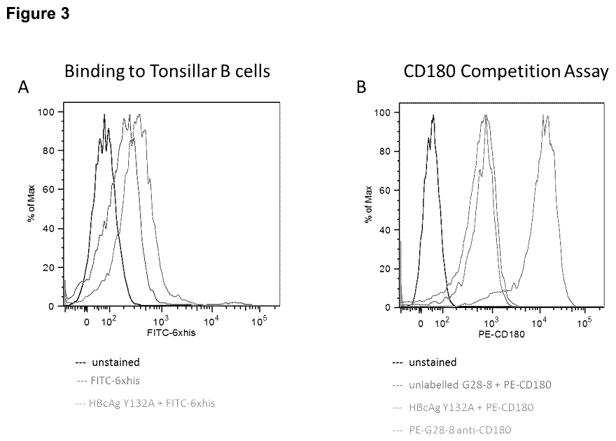

FIG. 3A shows that human CD20+ tonsillar B cells (10⁶) were incubated in 96 well round bottom plates with PBSA (PBS w/0.2% BSA+0.2% NaN3) media only (green) or with PBSA with the following His tagged recombinant protein containing the light and heavy chains of G28-8 anti-human CD180 (LH) and G28-8LH-HBcAgY132A-His (blue), at 10 μg/ml. After a 40 min incubation on ice, the cells were washed twice (centrifuged at 1200 rpm, 4 min). Then 100 μl of PBSA+5 μl a fluorescein (FITC)-conjugated anti 6×His (FITC-6×-His epitope tag ThermoScientific MA1-81891) were added to the wells, and after a 40 min incubation on ice, cells were washed twice and the level of fluorescence measured by flow cytometry shown on abscissa (log scale). Unstained cells shown in black. The recombinant protein bound to B cells as shown by binding being above the FITC control, demonstrating binding to CD180 expressed on B cells.

As a second test for binding, a competition assay was performed (FIG. 3B). For 25 negative and positive controls human B cells were either unstained (black) or stained with R-phycoerytherin G28-8 anti-CD180 (G28-8-PE, purple), respectively. Other tubes of B cells were first treated with 2.5 μg of either G28-8 mAb (green) or LH-HBcAgY132A-His (blue), for 40 min on ice, after which 10 μg G28-8-PE was added. After an incubation of 40 min on ice, cells were washed twice and analyzed by flow cytometry. As expected G28-8 mAb blocked the binding of G28-8-PE and reduced the level of fluorescence detected (green). G28-8LH-HBcAgY132A-His (blue) was as effective as the native G28-8 mAb at blocking binding, demonstrating that it bound effectively to human B cells and recognized CD180 specifically.

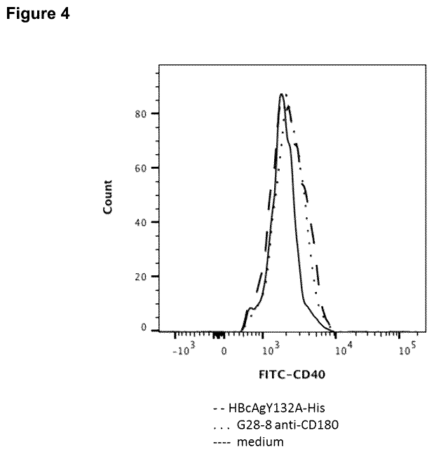

The ability of G28-8LH-HBcAgY132A-His to upregulate CD40 expression was then tested to evaluate its functional activity. Er-blood mononuclear cells enriched for B cells were incubated for 24 hrs at 37° C. with either media (black line) or 10 μg/ml of G28-8 (dotted line) or 10 μg/mIG28-8LH-HBcAgY132A-His. Samples were washed twice with PBSA, stained with mAb specific for CD20 (Pacific Blue Biolegend) and CD40 (FITC BD BioSciences) and evaluated for CD40 and CD20 expression using flow cytometry. FIG. 4 shows CD40 expression of gated CD20⁺ B cells. Both G28-8 and G28-8LH-HBcAgY132A-His upregulated CD40 expression to a similar extent, confirming that G28-8LH-HBcAgY132A-His was functionally active.

### EXAMPLE 3

### Induction by G28-8LH-HBcAgY132A-His Vaccine of HBcAg-specific IgG Antibody Responses in Rhesus Macaques

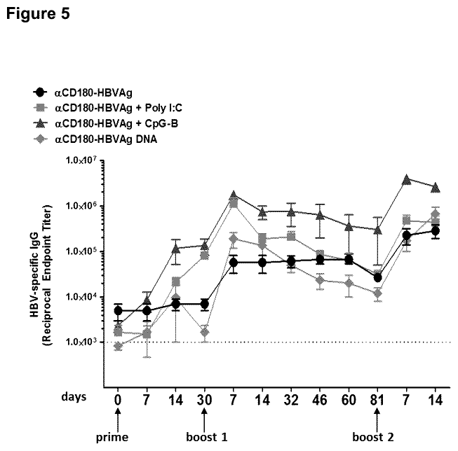

The ability of G28-8LH-HBcAg132A-His to induce humoral and cellular immune responses was examined in a vaccination experiment in rhesus macaques (Macaca mulatta). Groups of rhesus macaques (N=3) were vaccinated subcutaneously with either: 1) 300 μg of G28-8LH-HBcAgY132A-His (αCD180-HBVAg) in 1 ml; 2) 300 μg of G28-8LH-HBcAgY132A-His (HBcAg-CD180) plus 1 mg of long chain poly I:C (InvivoGen, San Diego) in 1 ml; 300 μg of G28-8LH-HBcAgY132A-His (HBcAg-CD180) plus 1 mg CpGB (Coley Pharmaceuticals) in 1 ml; or 4) 16 micrograms of G28-8LH-HBcAgY132A-His encoding plasmid DNA (HBcAg-CD180 DNA) coated onto 1 micron gold beads at a rate of 2.0 ug DNA per mg gold particles. The particles were then injected intracellularly into the epidermis of the skin using a gene gun (GG) as described.⁴¹ Animals were vaccinated on days 0, 30 and 81, and on days 0, 7, 14, 30 (time-points after first dose), 44, 62, 76, 90 (time-points after second dose), 111, 118, 125, 139 and 164 (time-points after 3^{rd} dose), serum and heparinized blood samples were obtained. Serum samples were assessed for IgG antibody responses to HBcAg by ELISA as follows: a) coating 96 well plates with 200 ng/well recombinant HBcAg (expressed in yeast); b) adding serial dilutions of serum samples (100 μl diluted in TBS+0.05% tween-20) starting with a 1:1000 dilution, followed by washing and adding HRP-anti-macaque IgG second step (Rockland, 1:5000 dilution). All four groups produced IgG after immunizations (FIG. 5). The antibody titers increased after each boost. The group receiving vaccine with a CpGB adjuvant had significantly higher IgG antibody responses compared to the other three groups.

### EXAMPLE 4

### Induction of HBcAg-Specific CD4 and CD8 T Cell Responses by G28-8LH-HBcAgY132A-His in Rhesus Macaques

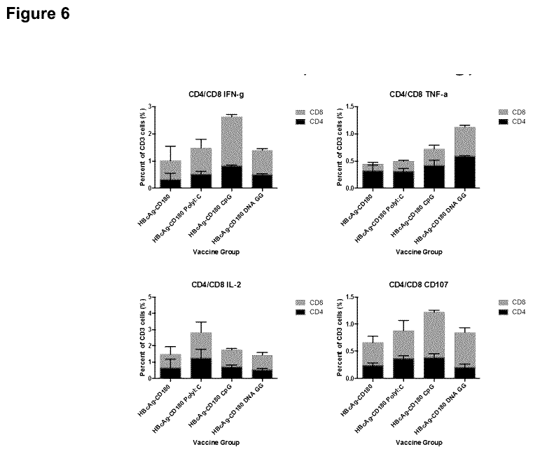

To determine the frequency of HBcAg-specific, intracellular cytokine-producing CD4 and CD8 T cells after vaccination of macaques, peripheral blood mononuclear cells were isolated from heparinized blood samples obtained from immunized macaques 28 days after the final dose on day 81 as noted in Example 3 and resuspended in growth media at defined concentrations (˜1.2 million cells/condition). Cells were plated together with either: Staphylococcal enterotoxin B (SEB, Toxin Technology, Sarasota) or PMA/lonomycin (positive controls); HBcAg peptide pools (Table 1, experimental); or DMSO (neg control) at the same concentration as in the peptide pools. After an incubation for 1 hr at 37° C. to initiate stimulation, brefeldin A was added to retain cytokines in the cells. Cells were then incubated for an additional 11-14 hrs, after which staining was initiated. First, cells were stained to detect surface markers: CD3, CD4, CD8 and CD107(a marker of cytolytic effector function), then cells were fixed, permeabilized and stained with antibodies that detect intracellular cytokines (IFNγ, TNFα or IL-2). Cells were then analyzed using an LSRII flow cytometer. Analyses were performed using PBMCs obtained 28 days after a second booster vaccination first treated as follows: Cells (1.2×10⁶) were stimulated in 96 well plates with 37 HBcAg peptides (Table 1) divided into 2 pools of n=18 and n=19 peptides at 2 μg/ml together with costimulatory anti-CD28 and anti-CD49d antibodies (5 μg/ml in total volume of 200 μl) for 1 hour at 37 C. Cells in control wells were incubated with medium only (negative control) or with SEB enterotoxin (5 μg/ml, positive control). Brefeldin A (Sigma) was added to wells at a final concentration of 0.05 μg/ml, and then cells were incubated for an additional 5 hours at 37 C, after which plates were wrapped in plastic and aluminum foil and incubated overnight at 4 C. Cells were then centrifuged and washed with PBS and stained with a live/dead cell stain (Invitrogen), incubated for 10 minutes (min) at room temperature (RT), washed and then stained with sets of chromophore-labeled monoclonal antibodies (mAbs) specific for cell surface markers included CD3-APC, CD4-PerCP Cy5.5, CD8-APC-Cy7, CD28-PECF594 and CD107a/b-FITC (all Becton Dickinson, BD) and CCR7-PerCPefluor710 (eBioscience) at 1:50 dilution, 50 μl. After a 30 min incubation in the dark at RT, cells were centrifuged and washed with PBS and then treated with BD Fixation/Permeabilization Solution (1×, Cytofix/Cytoperm™ kit BD # 554714), incubated in the dark for 20 min at RT, and washed twice with 200 μl/well of 1×BD Perm/Wash™ buffer. After removal of this wash buffer, cells were stained with 100 μl of a combination of TNFα-PE Cy7 1:20 (BD), IL-2 PE 1:10 (Biolegend) or IFNγ-V450 1:20 (Biolegend), mixed, and incubated for 60 min at 4 C. Cells were then washed with 1×BD Perm/Wash™ Buffer and fixed with 2% paraformaldehyde in PBS for at least 1 hour at 4 C before analysis using an LSR II flow cytometer. FIG. 6 shows the net percent of CD3+CD4+ cells (black) or CD3+CD8+ cells (gray) that expressed IFNγ, TNFα, IL-2 or CD107 for each vaccinated group after background levels (percent frequencies measured in unstimulated DMSO groups) are subtracted. Vaccination with the G28-8LH-HBcAgY132A-His protein only induced clear CD4 and CD8 T cell responses including IFNγ+ and CD107+CD8+ T cell responses that are known to contribute to cytotoxic T cell activity.

TABLE 1

| Peptide number | NH2 terminal (left) |
|---|---|
| 1 (SEQ ID NO: 26) | SKLCLGWLWGMDIDP |
| 2 (SEQ ID NO: 27) | LGWLWGMDIDPYKEF |
| 3 (SEQ ID NO: 28) | WGMDIDPYKEFGASV |
| 4 (SEQ ID NO: 29) | IDPYKEFGASVELLS |
| 5 (SEQ ID NO: 30) | KEFGASVELLSFLPS |
| 6 (SEQ ID NO: 31) | ASVELLSFLPSDFFP |
| 7 (SEQ ID NO: 32) | LLSFLPSDFFPSIRD |
| 8 (SEQ ID NO: 33) | LPSDFFPSIRDLLDT |
| 9 (SEQ ID NO: 34) | FFPSIRDLLDTASAL |
| 10 (SEQ ID NO: 35) | IRDLLDTASALYREA |
| 11 (SEQ ID NO: 36) | LDTASALYREALESP |
| 12 (SEQ ID NO: 37) | SALYREALESPEHCS |
| 13 (SEQ ID NO: 38) | REALESPEHCSPHHT |
| 14 (SEQ ID NO: 39) | ESPEHCSPHHTALRQ |
| 15 (SEQ ID NO: 40) | HCSPHHTALRQAILC |
| 16 (SEQ ID NO: 41) | HHTALRQAILCWGEL |
| 17 (SEQ ID NO: 42) | LRQAILCWGELMNLA |
| 18 (SEQ ID NO: 43) | ILCWGELMNLATWVG |
| 19 (SEQ ID NO: 44) | GELMNLATWVGSNLE |
| 20 (SEQ ID NO: 45) | NLATWVGSNLEDPAS |
| 21 (SEQ ID NO: 46) | WVGSNLEDPASRELV |
| 22 (SEQ ID NO: 47) | NLEDPASRELVVSYV |
| 23 (SEQ ID NO: 48) | PASRELVVSYVNVNM |
| 24 (SEQ ID NO: 49) | ELVVSYVNVNMGLKI |
| 25 (SEQ ID NO: 50) | SYVNVNMGLKIRQLL |
| 26 (SEQ ID NO: 51) | VNMGLKIRQLLWFHI |
| 27 (SEQ ID NO: 52) | LKIRQLLWFHISCLT |
| 28 (SEQ ID NO: 53) | QLLWFHISCLTFGRE |
| 29 (SEQ ID NO: 54) | FHISCLTFGRETVLE |
| 30 (SEQ ID NO: 55) | CLTFGRETVLEYLVS |
| 31 (SEQ ID NO: 56) | GRETVLEYLVSFGVW |
| 32 (SEQ ID NO: 57) | VLEYLVSFGVWIRTP |
| 33 (SEQ ID NO: 58) | LVSFGVWIRTPPAYR |
| 34 (SEQ ID NO: 59) | GVWIRTPPAYRPPNA |
| 35 (SEQ ID NO: 60) | RTPPAYRPPNAPILS |
| 36 (SEQ ID NO: 61) | AYRPPNAPILSTLPE |
| 37 (SEQ ID NO: 62) | PNAPILSTLPETTVV |

### EXAMPLE 5

### Induction of HBcAg-Specific IgG Antibody Responses in Rhesus Macaques by G28-8LH-HBcAgY132A-His Vaccine but not by HBcAgY132A-His Only

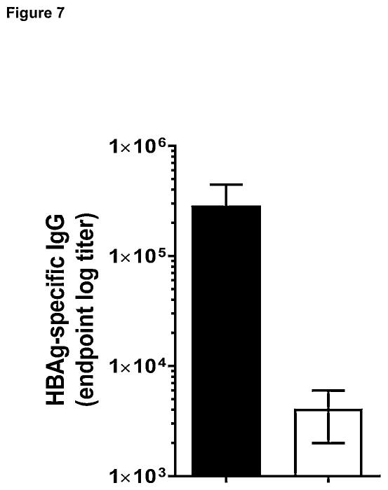

Whether or not targeted of HBcAg132A-His to CD180 was required to induce a humoral immune response was examined in a vaccination experiment in rhesus macaques (Macaca mulatta). Two groups of rhesus macaques (N=3) were vaccinated subcutaneously with either: 1) 300 μg of G28-8LH-HBcAgY132A-His (aCD180-HBVAg) in 1 ml; or 2) 150 μg of HBcAgY132A-His, which was equivalent to the amount of HBcAgY132A-His used in Group 1 where it was attached to G28-8LH. Animals were vaccinated on days 0 and 30 and on days 0 (prebleed) and 44 (14 days after second dose) serum and heparinized blood samples were obtained. Serum samples were assessed for IgG antibody responses to HBcAgY132A by ELISA as follows: a) coating 96 well plates with 200 ng/well recombinant HBcAgY132A (expressed in CHO cells as in FIG. 2); b) adding serial dilutions of serum samples (100 μl diluted in TBS+0.05% tween-20) starting with a 1:1000 dilution, followed by washing and adding HRP-anti-macaque IgG second step (Rockland, 1:5000 dilution). Only macaques in Group 1 produced IgG after immunizations (FIG. 7). The antibody titers in Group 2 were similar to titers in prebleed serum samples (not shown). Thus, targeting to CD180 via G28-8LH-HBcAgY132A-His is required to induce strong HBcAg-specific IgG antibody responses.

### LITERATURE CITED

1. DiMettle MA, Watts NR, Stahl SJ, Grimes JM, Steven AC, Stuart DI, Wingfield PT. Antigenic switching of hepatitis B virus by alternative dimerization of the capsid protein. Structure. 2013. 21:133-42.
2. Kim H.N., et al., Hepatitis B vaccination in HIV-infected adults: current evidence, recommendations and practical considerations. International journal of STD & AIDS, 2009. 20:595-600.
3. Kubba, A.K., et al., Non-responders to hepatitis B vaccination: a review. Communicable disease and public health/PHLS, 2003. 6:106-12.
4.Ott, J.J., et al., Global epidemiology of hepatitis B virus infection: new estimates of age-specific HBsAg seroprevalence and endemicity. Vaccine 2012. 30:2212-9.
5. Weinbaum CM, Williams I, Mast EE, et al. Recommendations for identification and public health management of persons with chronic hepatitis B virus infection. Centers for Disease Control and Prevention (CDC). MMWR Recomm Rep. 2008. 57(RR-8):1-20.
6. Wasley A, Kruszon-Moran D, Kuhnert W, et al. The prevalence of hepatitis B virus infection in the United States in the era of vaccination. J Infect Dis. 2010. 202:192-201.
7. Mitchell T, Armstrong GL, Hu DJ, Wasley A, Painter JA. The increasing burden of imported chronic hepatitis B-United States, 1974-2008. PLoS One. 2011. 6:e27717.
8. Hepatitis B vaccines. Releve epidemiologique hebdomadaire/Section d'hygiene du Secretariat de la Societe des Nations=Weekly epidemiological record/Health Section of the Secretariat of the League of Nations, 2004. 79:255-63.
9. Perz JF et al., The contributions of hepatitis B virus and hepatitis C virus infections to cirrhosis and primary liver cancer worldwide. J Hepatol 2006. 45:529-38.
10. Lavanchy, D., Hepatitis B virus epidemiology, disease burden, treatment, and current and emerging prevention and control measures. J Viral Hep 2004. 11:97-107.
11. Kim WR, Epidemiology of hepatitis B in the United States. Hepatology, 2009. 49: S28-34.
12. Wang L, Zou ZQ, Liu CX, Liu XZ. Immunotherapeutic interventions in chronic hepatitis B virus infection: a review. J Immunol Methods. 2014 May;407:1-8.
13. Thai H, Campo DS, Lara J, et al. Convergence and coevolution of hepatitis B virus drug resistance. Nat Commun. 2012. 3:789.
14. Menéndez-Arias L, Alvarez M, Pacheco B. Nucleoside/nucleotide analog inhibitors of hepatitis B virus polymerase: mechanism of action and resistance. Curr Opin Virol. 2014. 8C:1-9.
15. Wiegand J, van Bömmel F, Berg T. Management of chronic hepatitis B: status and challenges beyond treatment guidelines. Semin Liver Dis 2010;30:361-377.
16. Luckhaupt SE, Calvert GM. Deaths due to bloodborne infections and their sequelae among health-care workers. Am J Ind Med. 2008. 51:812-24.
17. Beck, J. and M. Nassal, Hepatitis B virus replication. World J Gastroenterol WJG, 2007. 13:48-64.
18. Maini MK, Boni C, Lee CK, Larrubia JR, Reignat S, Ogg GS, et al. The role of virus-specific CD8(+) cells in liver damage and viral control during persistent hepatitis B virus infection. J Exp Med 2000;191:1269-1280.
19. Akbar SM, Chen S, Al-Mahtab M, Abe M, Hiasa Y, Onji M. Strong and multi-antigen specific immunity by hepatitis B core antigen (HBcAg)-based vaccines in a murine model of chronic hepatitis B: HBcAg is a candidate for a therapeutic vaccine against hepatitis B virus. Antiviral Res 2012;96:59-64.
20. Cao W, Qiu Z, Zhu T, Li Y, Han Y, Li T. CD8+ T cell responses specific for hepatitis B virus core protein in patients with chronic hepatitis B virus infection. J Clin Virol. 2014. 61:40-6.
21. Penna A, Chisari FV, Bertoletti A, et al. Cytotoxic T lymphocytes recognize an HLA-A2-restricted epitope within the hepatitis B virus nucleocapsid antigen. J Exp Med. 1991. 174:1565-70.
22. Sun L, Zhang Y, Zhao B, et al. A new unconventional HLA-A2-restricted epitope from HBV core protein elicits antiviral cytotoxic T lymphocytes. Protein Cell. 2014. 5:317-27.
23. Valentine MA, Clark EA, Shu GL, Norris NA, Ledbetter JA. Antibody to a novel 95-kDa surface glycoprotein on human B cells induces calcium mobilization and B cell activation. J Immunol. 1988. 140:4071-8.
24. Miyake, K., et al., Murine B cell proliferation and protection from apoptosis with an antibody against a 105-kD molecule: unresponsiveness of X-linked immunodeficient B cells. J Exp Med 1994. 180:1217-24.
25. Miyake, K., et al., RP105, a novel B cell surface molecule implicated in B cell activation, is a member of the leucine-rich repeat protein family. J Immunol 1995. 154:3333-40.
26. Alving CR, Peachman KK, Rao M, Reed SG. Adjuvants for human vaccines. Curr Opin Immunol. 2012. 24:310-5.
27. Shimazu, R., et al., MD-2, a molecule that confers lipopolysaccharide responsiveness on Toll-like receptor 4. J Exp Med 1999. 189:1777-82.
28.Hebeis, B., et al., Vav proteins are required for B-lymphocyte responses to LPS. Blood, 2005. 106:635-40.
29. Hebeis, B.J., et al., Activation of virus-specific memory B cells in the absence of T cell help. J Exp Med 2004. 199:593-602.
30. Yazawa, N., et al., CD19 regulates innate immunity by the toll-like receptor RP105 signaling in B lymphocytes. Blood, 2003. 102:1374-80.
31. Chaplin, J.W., et al., Anti-CD180 (RP105) activates B cells to rapidly produce polyclonal Ig via a T cell and MyD88-independent pathway. J Immunol, 2011. 187:4199-209.
32. Chaplin JW, Chappell CP, Clark EA. Targeting antigens to CD180 rapidly induces antigen-specific IgG, affinity maturation and immunologic memory. 2013. J Exp Med 210:2135-46.
33. Ramos, H.J. and M. Gale, Jr., RIG-I like receptors and their signaling crosstalk in the regulation of antiviral immunity. Curr Opin Virol, 2011. 1:67-76.
34. Maxon ER, Siegrist CA. The next decade of vaccines: societal and scientific challenges. Lancet. 2011. 378:348-59.
35. Liang Y et al., Predictors of relapse in chronic hepatitis B after discontinuation of anti-viral therapy. Aliment Pharmacol Ther, 2011. 34:344-52.
36. Suthar MS, Diamond MS, Gale M, Jr. West Nile virus infection and immunity. 2013. Nat Rev Microbiol 11:115-128.
37. Coffman RL, Sher A, Seder RA. Vaccine adjuvants: putting innate immunity to work. Immunity. 2010. 33:492-503.
38. Bourne CR, Katen SP, Fulz MR, Packianathan C, Zlotnick A. A mutant hepatitis B virus core protein mimics inhibitors of icosahedral capsid self-assembly. Biochemistry. 2009. 48:1736-1742.
39. Packianathan C, Katen SP, Dann CE, 3rd, Zlotnick A. Conformational changes in the hepatitis B virus core protein are consistent with a role for allostery in virus assembly. J Virol. 2010. 84:1607-1615.
40. Clark EA, Shu GL, Luscher B, Draves KE, Banchereau J, Ledbetter JA, Valentine MA. Activation of human B cells. Comparison of the signal transduced by IL-4 to four different competence signals. J Immunol. 1989. 143:3873-80.
41. Loudon PT, Yager EJ, Lynch DT, Narendran A, Stagnar C, Franchini AM, Fuller JT, White PA, Nyuandi J, Wiley CA, Murphey-Corb M, Fuller DH. GM-CSF increases mucosal and systemic immunogenicity of an H1N1 influenza DNA vaccine administered into the epidermis of non-human primates. PLoS One. 2010. 5:e11021.

1. A method for treating or limiting the development of a hepatitis-B virus (HBV)-related disorder, comprising administering to an individual in need thereof an amount effective to treat or limit development of the HBV-related disorder of a composition comprising a polypeptide, wherein the polypeptide comprises an amino acid sequence at least 90% identical to the amino acid sequence of SEQ ID NO:3 residues 1-418.

2. The method of claim 1, wherein the individual is at risk of infection with HBV.

3. The method of claim 2, wherein the individual is less than one month old, at least 65 years old, immunodeficient, and/or immunosuppressed.

4. The method of claim 1, wherein the individual is at risk of an HBV-related disorder selected from the group consisting of hepatitis, hepatitis-related disease, fulminant hepatitis, cirrhosis, and hepatocellular carcinoma.

5. The method of claim 1, wherein the individual is infected with hepatitis B virus.

6. The method of claim 5, wherein the individual has a hepatitis B-related disease, select4d from the group consisting of hepatitis, hepatitis-related disease, fulminant hepatitis, cirrhosis, and/or hepatocellular carcinoma.

7. The method of claim 1, wherein the polypeptide comprises the amino acid sequence of SEQ ID NO: 2, 3, 5, 6, 8, 9, 11, 12, 18, or 19.

8. The method of claim 1, wherein the polypeptide comprises the amino acid sequence of SEQ ID NO:3 residues 1-418.

9. The method of claim 1, wherein the polypeptide comprises the amino acid sequence of SEQ ID NO:3.

10. The method of claim 1, wherein the composition further comprises an adjuvant.

11. A method for treating or limiting the development of a hepatitis-B virus (HBV)-related disorder, comprising administering to an individual in need thereof an amount effective to treat or limit development of the HBV-related disorder of an expression vector comprising a nucleic acid encoding a polypeptide comprising an amino acid sequence at least 90% identical to the amino acid sequence of SEQ ID NO:3, residues 1-418, wherein the nucleic acid is operatively linked to a promoter.

12. The method of claim 11, wherein the polypeptide comprises the amino acid sequence of SEQ ID NO: 2, 3, 5, 6, 8, 9, 11, 12, 18, 19, 20, or 21.

13. The method of claim 11, wherein the polypeptide comprises an amino acid sequence at least 90% identical to the amino acid sequence of SEQ ID NO:3, residues 1-418.

14. The method of claim 11, wherein the polypeptide comprises the amino acid sequence of SEQ ID NO:3, residues 1-418.

15. The method of claim 11, wherein the polypeptide comprises the amino acid sequence of SEQ ID NO:2, residues 1-438.

16. The method of claim 11, wherein the polypeptide comprises the amino acid sequence of SEQ ID NO:2, residues 1-438.

17. The method of claim 11, wherein the individual is at risk of infection with HBV.

18. The method of claim 17, wherein the individual is less than one month old, at least 65 years old, immunodeficient, and/or immunosuppressed.

19. The method of claim 11, wherein the individual is at risk of an HBV-related disorder selected from the group consisting of hepatitis, hepatitis-related disease, fulminant hepatitis, cirrhosis, and hepatocellular carcinoma.

20. The method of claim 11, wherein the individual is infected with hepatitis B virus.

21. The method of claim 20, wherein the individual has a hepatitis B-related disease, selected from the group consisting of hepatitis, hepatitis-related disease, fulminant hepatitis, cirrhosis, and/or hepatocellular carcinoma.
